(19) United States
(12) Patent Application Publication
(10) Pub. No.: US 2015/0359878 A1
(43) Pub. Date: Dec. 17, 2015

(54) USE OF REPLICATION DEFICIENT HSV-1 AS A VACCINE VECTOR FOR THE DELI VARY OF HIV-1 TAT ANTIGEN

(71) Applicant: VAXXIT SRL, Roma (IT)

(72) Inventors: Peggy MARCONI, Ferrara (IT); Antonella CAPUTO, Ferrara (IT); Riccardo GAVIOLI, Ferrara (IT); Barbara ENSOLI, Roma (IT); Roberto MANSERVIGI, Ferrara (IT)

(21) Appl. No.: 14/762,386

(22) PCT Filed: Jan. 22, 2014
(86) PCT No.: PCT/EP2014/051277

§ 371 Date: Jul. 21, 2015

(30) Foreign Application Priority Data
Jan. 22, 2013 (GB) 1301119.2

(51) Int. Cl. A61K 39/21; A61K 39/04; C12N 7/00

(52) U.S. Cl. CPC A61K 39/21; C12N 7/00; A61K 39/04; C12N 2740/16034; C12N 2710/16643; A61K 2039/70

(57) ABSTRACT

A vaccine for a DNA virus, especially HSV1, comprises an expression vector for HIV1 Tat, wherein the vector is an avirulent form of said DNA virus, and elicits cellular responses to cryptic epitopes of the DNA virus, as well as eliciting a detectable IgG response.

The present invention relates to vaccines comprising HIV1 Tat, their components, and methods of manufacture.

Clinical trials using HSV envelope glycoproteins failed in prevention and protection. Together with the recent observation that the epitopes recognised by T-effector cells of symptomatic patients are different from those recognised by the T-effector cells of asymptomatic patients who have a decrease of recurrent HSV infections (1), it can be seen that the effectiveness of HSV vaccines depends on their ability to induce the appropriate cellular arm of immunity. A vaccine able to promote an adequate T-cell response against these epitopes is, therefore, necessary in order to be able to treat recurrent herpes disease (1, 2). In this regard, identification of HSV vectors that can promote signals favouring the emergence of a Th1 immune response not only against dominant epitopes but also against subdominant epitopes, that are crucial to halt viral reactivation, is a key point for the development of novel vaccine strategies against HSV infection (3-11).

The World Health Organization (WHO) declared tuberculosis (TB) a global health emergency, and the Global Plan to stop tuberculosis aims to save 14 million lives between 2006-2015 and has, as a major goal, the eradication of TB by developing new vaccine strategies that can be effective for the whole population (http://www.amref.it).

The challenge to eradicate TB has led the scientific community to an escalation in the search for new adjuvants to associate with new recombinant Bacille Calmette Guerin (BCG) vaccines, for increased effectiveness, and to find new vaccines that can be used side by side with the existing BCG vaccine {Delogu, 2009; Bastos, 2009; Vordermeier, 2009}. One aim is to find new vaccines that can be used as boosters after a primary vaccination with BCG to increase the response against TB at all ages, and for those already sensitized subjects and against all stages of TB (latent, pulmonary, extra pulmonary) {Beveridge, 2007; Roediger, 2008}.

Recombinant DNA techniques to advance the identification of new antigens and approaches for TB vaccination have been explored {Denis, 1998; Delogu, 2000; Ulmer, 1998}. Preliminary studies in mice have demonstrated that vaccination with DNA expressing Mt antigens is able to increase considerably the T cell-mediated immune response repertoire characterized by CD4+ and CD8+ cells {Denis, 1998}. Experimental data have shown that genetic immunization with constructs that express secreted proteins, such as Ag85A/B, ESAT-6, and Mpt 64/63/83, gives significant protection in treated animals {Delogu, 2002; Sali, 2008; Derrick, 2004}. Moreover, it has been demonstrated that immunization with plasmids that express a combination of the above mentioned proteins are more effective and give better and higher levels of protection in comparison with monovalent DNA vaccines {Derrick, 2004}.

To increase the efficacy of genetic vaccination, it appears to be promising to employ modified viruses such as alphavirus, adenovirus, vaccinia virus as a vectors to express genes encoding Mt protective antigens and/or cytokines {Vordermeier, 2009; Mu, 2009; Hashimoto, 2008; Roediger, 2008; Xing, 2005}.

Chronic immune activation leading to progressive immune exhaustion is a central feature of HIV pathogenesis (12-15). The HIV1 Tat protein, which is essential for virus replication and efficient virus gene expression, has a variety of other activities affecting the delicate balance between stimulatory and inhibitory host factors of the immune system that contribute to the immune dysregulation observed in HIV-infected subjects (16-27). Recently, results from a phase II clinical trial using a HIV1 Tat protein based-vaccine have shown that anti-Tat antibodies induced by vaccination correlate with an overall improvement of immune functions in HIV infected patients, including the restoration of T cell activity, the normalisation of the balance between the different immune cells populations and the decrease of immune activation levels (28).

The reconstitution of the immune system due to the blockage of Tat by anti-Tat antibodies supports a key role of Tat in the immune dysregulation associated with HIV/AIDS natural infection (29-31). Several studies attempting to evaluate the role of the Tat protein on immune cell functions sustain this hypothesis, even if the mechanism of Tat-induced immune dysregulation in HIV-infected patients is still under debate due to some, at least apparent, inconsistencies demonstrated by different works. Indeed, various immunosuppressive Tat activities have been described, including the inhibition of T cell proliferation and the down-regulation of receptors that could impact the immune response to HIV (16, 23, 27).

On the other hand, other studies have suggested that Tat possesses immunostimulatory activities favouring T cell activation and, as a consequence, general immune activation (20, 24, 26).

Different experimental conditions, in vitro models, and recombinant Tat proteins, may explain these conflicting results. Moreover, the context in which Tat operates in HIV-infected patients is very complex and, often, experimental settings do not take into consideration assorted factors, such as the unavailability of soluble Tat due to its sequestration in certain tissues, or the complex cytokine milieu and different types of stimuli to which immune cells are exposed (32) (33, 34).

Surprisingly, after inoculation of an attenuated recombinant HSV1-Tat in mice, impairment of immune responses against HSV1 was not observed but, rather, the expression of Tat within the recombinant virus induced cellular and humoral immune responses against HSV1, which was in direct contrast with the control virus (HSV1-LacZ) which did not express Tat and that did not induce any such response. Mice that had received HSV1-Tat were also protected from challenge from a lethal dose of wild-type HSV1.

Thus, in a first aspect, the present invention provides a vaccine for a DNA virus, comprising an expression vector for HIV1 Tat, wherein the vector is a compromised form of said DNA virus, especially an attenuated or avirulent form.

The vector may be an RNA vector or a DNA vector. Where the vector is an RNA vector, then this may be provided as a reverse transcript of the avirulent form of the DNA virus. However, it is preferred that the vector does not integrate into the host DNA and, as such, it is preferred that the vector is a DNA vector.

The vaccine may be for any mammal, although humans are preferred. Preferred non-human mammals are commercially important mammals, such as farm animals, race horses, and those kept in zoological gardens.

The DNA viruses include various orders, families, and unassigned families. The Caudovirales includes the families Myoviridae, Podoviridae, Siphoviridae. The order Herpesvirales includes the families Alloherpesvirdae, Herpersviridae, and Malacoherpesviridae. The order Ligamenvirales includes the families Lipothrixviridae, and Rudiviridae. The following families belong to no specific order; Adenovirdae, Ampullaviridae, Ascoviridae, Asfarviridae, Baculoviridae, Bicaudaviridae, Clavaviridae, Corticoviridae, Fuselloviridae, Globuloviridae, Guttaviridae, Hytrosaviridae, Iridoviridae, Miminviridae, Nimaviridae, Papillomaviridae, Phycodnaviridae, Plasmaviridae, Polydnaviruses, Polyomaviridae, Poxviridae, and Tectiviridae.

A preferred order is the Herpesvirales. A preferred family for use in the present invention is the Herpesviridae, and particularly herpes simplex virus type 1, or HSV-1.

The vector of the present invention comprises a compromised form of a DNA virus. By “compromised” is meant a form of the virus that is not capable of the full aetiology and pathology of the naturally occurring virus, and will often be rendered incapable in some essential aspect, such as reproduction, toxicity, or transmission. Preferred compromised viruses are attenuated and/or avirulent forms. By “attenuated” is meant that a replication-competent vector form of the virus is capable of replication, but is less pathogenic. By “avirulent” is meant that a replication-defective form of the virus is not capable of a virulent infection in an individual, i.e. where the virus cannot be transferred to another individual by the normal route of infection associated with the relevant DNA virus. It is also preferred that, where the DNA virus is associated with external symptoms, that such symptoms are either absent, substantially absent, or largely unnoticeable, although it will be noted that vaccinations may often be accompanied by brief rashes and/or briefly elevated temperature and other transient symptoms otherwise associated with a virulent infection. It will be appreciated that the terms “compromised”, “attenuated” and “avirulent” are used interchangeably herein, unless otherwise apparent from their context.

In one embodiment, we have constructed viral vectors based on Herpes simplex type 1 (HSV-1) virus. It is an advantage of such vectors that they have sufficient characteristics to carry vaccine antigens alone or in combination. It is preferable that the HSV vectors for prophylaxis against infections have one or more of the following characteristics: a) are genetically stable, b) are incapable of replicating in the CNS, c) are unable to reactivate, and d) are immunogenic and protective against diseases.

As noted above, a preferred DNA virus is the HSV1 virus, and this will be used herein to illustrate the present invention. It will be understood that, where the term “HSV1” is used, then this equally applies to other DNA viruses of the present invention, unless otherwise apparent.

In order to ensure that the virus is attenuated or avirulent, it is preferred to integrate DNA encoding HIV1 Tat into a location on the HSV1 genome that encodes a significant function, or protein. In HSV1, this may suitably be the viral host shutoff protein (vhs).

It is generally preferred that the vector is a form of the virus and that it is encapsulated. This ensures that, when the vector is delivered to the patient, the vector is delivered into the cellular mechanism in order to be able to express both HIV1 Tat and the other proteins encoded by the vector. Accordingly, it will be understood that, where HIV1 Tat has been introduced into the genome of the virus, then it is preferred to excise an appropriate amount of DNA from the virus, which is replaced by DNA encoding HIV1 Tat, thereby enabling the vector to fit within the space available in the viral capsid.

The DNA that is excised may include all or part of the function that is being replaced. Where a live, attenuated virus is being transformed, then it may not always be necessary to replace a function, and any part of the vector can be replaced, provided that the vector can still be expressed in situ.

The DNA to be excised may be of any length, and include any number of functions and/or significant proteins, provided that the vector can be introduced into the cellular mechanism and be expressed therein. The HIV1 Tat-encoding DNA may be in any suitable form for expression in the vector. At its simplest, cDNA may be introduced directly into the vector in operable relationship with a suitable promoter therefor. One such promoter is the HSV1 ICP0 promoter, as described hereinbelow. The promoter may already be present in the vector, or may be inserted in the vector separately from, or together with, the Tat-encoding sequence. For example, it is possible to construct a cassette comprising the Tat-encoding sequence and the promoter. This cassette can then be inserted into the vector, as desired. A Tat expression cassette is preferably any that is suitable to ensure the expression of the tat gene within the host cell to produce Tat in a biologically active form.

It will be appreciated that, whilst it is preferred to use the naturally occurring sequence of HIV1-Tat, the present invention envisages that any variant or mutant of the naturally occurring HIV1-Tat can be employed in the present invention, provided that such variant or mutant has substantially similar, or better, adjuvant effects as naturally occurring HIV1-Tat. In this regard, the adjuvant effects include, at least, the ability to elicit a detectable IgG response against the DNA virus of the vaccine.

The preferred sequence of Tat for use in the present invention is SEQ ID NO:1:

ATGGAGCCAGTAGATCCTAGACTAGAGCCCTGGAAGCATCCAGGAAGTCA
GCCTAAAACTGCTTGTACCAATTGCTATTGTAAAAAGTGTTGCTTTCATT
GCCAAGTTTGTTTCATAACAAAAGCCTTAGGCATCTCCTATGGCAGGAAG
AAGCGGAGACAGCGACGAAGACCTCCTCAAGGCAGTCAGACTCATCAAGT
TTCTCTATCAAAGCAACCCACCTCCCAATCCCGAGGGGACCCGACAGGCC
CGAAGGAATAG

With the preferred peptide sequence being SEQ ID NO:2:

MEPVDPRLEPWKHPGSQPKTACTNCYCKKCCFHCQVCFITKALGISYGRK
KRRQRRRPPQGSQTHQVSLSKQPTSQSRGDPTGPKE.

Attenuated or avirulent forms of the DNA virus include, but are not limited to, replication-deficient forms of the virus, attenuated forms of the virus, and forms of the virus modified, for example, to prevent integration of the viral genome into the host genome.

Once the vector has been created, it may be multiplied up for use in vaccines by incubation in a permissive cell culture that allows the vector to multiply and be encapsulated. For example, where the vector does not encode any capsid proteins, or is deficient in one capsid protein, the host cell culture may encode any capsid protein that is missing. Thus, whilst the resulting viral progeny is fully capable of infection, it is not capable of a full replication cycle in situ. In other forms of avirulent virus, a full replication cycle is envisaged, but wherein one or more virulent functions has been disabled, such as coat assembly.

In one embodiment, one or more functions enabling the virus to hide from the immune system are incapacitated, thereby ensuring that one or more known treatments for said virus can have a viricidal effect, if used on the vector of the invention.

Where the vector is in the form of a viral particle, then this may be incorporated into a vaccine preparation in any manner suitable, and as known in the art for the preparation of such vaccines. The viral load of such vaccines is well-known to those skilled in the art, and is generally dependent on the age, sex, and general health of the patient, as well as the infective ability and immunogenicity of the vector.

The vaccine may be in any suitable form, although eye drops and injectable formulations are generally preferred, the latter being, for example, in the form of intramuscular, intraperitoneal, subdermal and intravenous formulations.

It will also be appreciated that the vector may encode other functions or proteins that it is desired to incorporate into the vector. These may be in the form of markers or further treatments. However, the space for such further inserts is generally limited by the space available in the capsid, and it will be appreciated that viral functions and proteins must generally be deleted to allow the incorporation of such further elements. In general, it is preferred to maintain as many of the original viral genetic elements as possible, to permit the maximum immunogenic effect.

In particular, as noted above, by incorporating HIV1-Tat into the viral genome, the resulting, expressed Tat is capable of eliciting immune responses to even the cryptic epitopes of the virus, thereby enabling the immune system of the host to be able subsequently to attack an HSV1 infection.

Vaccines of the present invention may be targeted to more than just the DNA virus forming the vector, and may comprise coding sequences for further immunogens, especially from other infectious agents. These may be selected from the DNA viruses noted above, RNA viruses, or pathogenic bacteria. In the latter instance, the vaccine of the invention may include antigens, optionally cryptic antigens, from, for example, and of the following species: Bacillus anthracis, Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis and Enterococcus faecium, Escherichia coli (generally), Enterotoxigenic Escherichia coli (ETEC), Enteropathogenic E. coli, E. coli O157:H7, Francisella tularensis, Haemophilus influenzae, Helicobacter pylori, Legionella pneumophila, Leptospira interrogans, Listeria monocytogenes, Mycobacterium leprae, Mycobacterium tuberculosis, Mycoplasma pneumoniae, Neisseria gonorrhoeae, Neisseria meningitidis, Pseudomonas aeruginosa, Rickettsia rickettsii, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Vibrio cholerae, and Yersinia pestis.

When targeting tuberculosis, for example, the vaccine may comprise one or more mycobacterial antigens selected from ESAT6, Ag85B, Mpt64-63-83, along with HIV1-Tat.

When vaccines of the present invention encode antigens from a secondary infectious agent, such as a pathogenic bacterium, then it is preferred that the DNA virus of the invention is HSV.

It will be appreciated that the HIV1-Tat may be expressed as a fusion protein with preferred antigens, whether from the target DNA virus or from a secondary infectious agent, such as a pathogenic bacterium, or both.

The present invention provides vectors as defined herein. In particular, there is provided an expression vector for HIV1 Tat, wherein the vector is a compromised form of a DNA virus, especially an attenuated or avirulent form of said virus, said vector optionally further being adapted to express one or more antigens of a secondary infectious agent.

There is further provided the use of such vectors in the treatment and/or prophylaxis of a condition associated with the said virus, said condition optionally being a disease condition.

There is further provided a method of treatment of a condition associated with a DNA virus, comprising administration of an effective amount of a vector or vaccine as defined herein to a patient in need thereof.

In order to examine the role of Tat in the dysregulation of the immune system in an in vivo model of viral infection, we generated a recombinant replication-competent Herpes simplex virus type 1 (HSV1) expressing the HIV1 Tat protein (HSV1-Tat). This vector contains a deletion in the UL41 locus of HSV1 encoding the viral host shutoff (vhs) protein (35, 36), which is replaced by the HIV1 tat coding sequence. This model allows observation, in vivo, of the role of Tat during triggering of the adaptive immune responses against HSV1 infection, and models physiological conditions where Tat is encoded by the viral genome and is secreted by infected cells.

The HIV1 Tat protein has a variety of activities modulating the immune system. To examine Tat-mediated immune modulation in murine models of viral infection, a recombinant replication-competent Herpes simplex virus type 1 (HSV1) expressing the HIV1 Tat protein (HSV1-Tat) was generated. Surprisingly, expression of Tat within the recombinant virus increases and broadens Th1-like and CTL responses against HSV1 immunodominant and subdominant T cell epitopes and elicits detectable IgG responses. In contrast, a similar attenuated HSV1 recombinant vector without Tat (HSV1-LacZ) induces lower T cell responses, which are directed only against the immunodominant epitopes, and does not generate measurable IgG serum responses. In addition, and also surprisingly, mice that received HSV1-Tat were also protected from challenge with a lethal dose of wild-type HSV1, demonstrating that vectors expressing Tat are useful as vaccines.

The HSV vaccines of the present invention may comprise viral particles that are replication defective and/or live attenuated, for example. Preferably, viral particles for use in the invention comprise a recombinant HSV backbone capable of expressing all, or a majority, of HSV immunogenic genes, together with Tat, such that the Tat protein is expressed together with HSV genes, rather than being given as a separate component of the vaccine formulation.

As noted above, after inoculation of HSV1-Tat into mice, we observed no dysregulation of immune responses against HSV1, against expectation. Instead, and surprisingly, expression of Tat by the recombinant virus induced cellular and humoral immune responses against HSV1, which responses were not elicited by a similar, control virus that did not express Tat (HSV1-LacZ), with the recombinant virus of the invention being able to protect mice from challenge with a lethal dose of wild-type HSV1.

HIV1 Tat expressed by replication-competent HSV1 vectors not only does not dysregulate the immune system against HSV infection, but increases and broadens the Th1-like and CTL responses against HSV1 immunodominant and subdominant T cell epitopes, as well as eliciting detectable IgG responses. A similar attenuated HSV1 recombinant vector without Tat (HSV1-LacZ) induces lower T cell responses, which are directed only against the immunodominant epitopes, and fails to generate measurable IgG serum responses Immunisation with vaccines of the invention is effective, and vaccines of the invention are capable of protecting BALB/c and C57BL/6 mice against HSV lesions and death after challenge with a lethal dose of wild type HSV1 virus.

Without being bound by theory, it is possible that the properties of the vaccines of the invention are due, at least partially, to the immunomodulatory properties of Tat previously reported by several groups. Different studies have demonstrated that the biologically active Glade B Tat protein (aa 1-86) targets immature dendritic cells (DCs), induces DC maturation and polarises the immune response to the Th1 pattern through transcriptional activation of TNF-alpha gene expression, leading to more efficient presentation of both allogeneic and heterologous antigens (17, 22). Tat induces changes in the subunit composition of the proteasome, which correlate to altered enzymatic activities and modulation of CTL epitope generation in virally-infected cells and broadens in vivo T cell responses against cryptic epitopes of a co-antigen which are not expressed, or only poorly expressed (19, 21).

In addition, Tat possesses auto-adjuvanticity and adjuvanticity to unrelated antigens with respect to humoral responses (37, 38) and favours protective immunity against Leishmania major (39). Furthermore, it has been shown that Tat upregulates the transcription factor T-bet which regulates Th1 differentiation as well as the class switch recombination to IgG2a of B cells (40-42).

We have demonstrated that Tat may be used in vaccination strategies against HSV infection for which, up to now, no efficient vaccine has been available (43).

The use of attenuated HSV-based vaccines is preferred. Replication-competent HSV-based vectors may be derived from attenuated viruses, in which genes responsible for virulence, but not essential for replication in cultured cells, have either been mutated or deleted (44, 45), and possess important properties: i) HSV does not integrate into the cellular genome, but remains in an episomal state, thereby preventing insertional mutagenesis in the host; ii) anti-herpetic drugs (acyclovir, forscanet) are already available, and could therefore be used to counteract any undesired local or systemic effects, should they occur; iii) live attenuated, HSV-1-based vectors, featuring deletion of the non-essential UL41 gene, directly infect dendritic cells, allowing their maturation, efficient antigen presentation necessary for T-cell priming and induction of strong CTL responses against the delivered genes, both in murine and simian models; iv) HSV backbone vectors are able to accommodate up to 50 kbp of exogenous DNA, thereby allowing co-expression of multiple antigens with genes encoding, for example, for immunostimulatory molecules, in a live vector, having advantages in multi-component vaccine formulations that may be desirable to provide an efficient immune response against a pathogen; and v) high-titre purified recombinant viral stocks are readily obtained in vitro and can be easily administered in vivo by systemic or mucosal routes.

Concerns over the use of attenuated HSV as a vector for vaccine application include the potential of HSV vectors to establish latency, reactivate, or recombine with virulent wild-type strains (46, 47), and may be avoided, for example, by defining and eliminating genes involved in neurovirulence, latency, or reactivation. For instance, to overcome some safety issues, the HSV1-Tat-based vector can be modified with other deletions on genes responsible for neurovirulence and down-modulation of antigen presentation (47). It is preferred that HSV-based vectors are free of functions employed by the virus to evade the immune system. At least two HSV gene products, the immediate early US12 gene product ICP47, and the viral host shutoff protein (vhs) encoded by the UL41 locus, are significant virulence factors due to their ability to disarm elements of the innate and adaptive host immune responses (48-51).

In particular, vhs is an endoribonuclease packaged into the tegument of mature HSV virions that, once delivered into the cytoplasm of newly infected cells, causes shutoffof host protein synthesis, disruption of preexisting polysomes, and degradation of host mRNAs (49), thus enhancing virus replication and accounting for the modest reduction in virus yield displayed by vhs mutants in cultured Vero cells (52, 53). Vhs also plays a critical role in HSV pathogenesis, as vhs mutants are severely impaired for replication in the corneas and central nervous systems of mice and cannot efficiently establish or reactivate from latency (54-56).

Live, attenuated HSV-based vectors have recently been demonstrated to be able to induce antibody or proliferative responses, regardless of pre-existing immunity to HSV, and satisfactory safety results have been obtained in independent preclinical and clinical studies with attenuated, replication-competent HSV recombinant vectors for cancer gene therapy (45) (http://clinicaltrials.gov/), thereby demonstrating that HSV-derived vectors are useful as effective means of vaccine delivery (57, 58).

The accompanying Figures are referred to hereinbelow and serve only to illustrate the present invention:

FIG. 1 shows a schematic representation of pBlueScript plasmids containing pr-lacZ or pr-tat cassettes, flanked by HSV UL41 flank sequences (A);

FIG. 2 shows an analysis of HSV1-specific T cell responses in C57BL/6 mice;
FIG. 3 shows an analysis of HSV1-specific T cell responses in BALB/c mice;
FIG. 4 shows an analysis of HSV1-specific T cell responses in C57BL/6 mice;
FIG. 5 shows an evaluation of anti-HSV1 specific antibody titres (IgG, IgG1, IgG2a) in sera of individual mice treated with HSV1-Tat or HSV1-LacZ;

FIG. 6 shows survival of BALB/c and C57BL/6 mice treated with HSV1-LacZ or HSV1-Tat following challenge with a lethal dose of HSV1;

FIG. 7 shows survival of C57BL/6 mice treated with HSV1-LacZ or HSV1-Tat following challenge with a lethal dose of HSV1;

FIG. 8 shows a schematic representation of BAC-HSVLuc genome (A). Schematic representation of HSVLucΔ27 vector (B). Schematic representation of HSVLucΔ27gJHE vector (C). Schematic representation of HSVLucΔ27gJTat vector (D);

FIG. 9 shows a schematic representation of the pcDNA3.1/Hygro+ TB5Ag plasmid;

FIG. 10 shows a schematic representation of the pB41tB5Ag plasmid;

FIG. 11 shows the schematic representation of the fusion protein pTB5Ag;

FIG. 12 shows a schematic representation of the SHtB5Ag/gJHE vector construction through recombination of pB41tB5Ag into UL41 locus containing the LacZ gene of S0ZgJGFP viral DNA;

FIG. 13 shows the schematic representation of SHtB5Ag/gJHE, S0Z/gJHTat and SHtB5Ag/gJtat;

FIG. 14 shows the biological activity of Tat protein; and

FIG. 15 shows the 8 TB peptides and 2 HSV were used to evaluate anti-TB and anti-HSV T cell responses respectively in BALB/c mice.

### EXPERIMENTAL

### Example 1

### Materials and Methods

### Cell Lines

Vero cells, a monkey kidney fibroblast cell line, and BALB/c cells, a fibroblast cell line derived from BALB/c mice, were grown in DMEM (Euroclone, Grand Island, N.Y.) supplemented with 10% FBS (Euroclone), 2 mM L-glutamine, 100 mg/ml penicillin and 100 U/ml streptomycin at 37° C. in 5% of CO₂ incubator.

### Generation of Plasmids and Attenuated Replication-Competent HSV1-Based Vectors

The plasmid pB41-lacZ contains the lacZ coding sequences (flanked at 5′ and 3′ ends by Pac I sites) inserted into the UL41 locus of HSV1 between UL41 flank fragments (HSV genomic positions 90145-91631 and 92230-93854) under the control of HSV1 ICP0 promoter (ICP0 pr), as previously described (35, 59). The recombinant, attenuated, replication-competent HSV1LacZ, wherein the UL41 gene is deleted by the insertion of the lacZ gene under the control of the HSV ICP0 pr, was generated by homologous recombination between wild-type HSV1 (strain LV) and plasmid pB41-lacZ. Briefly, Vero cells were co-transfected with HSV1 (LV strain) DNA and the pB41-lacZ plasmid DNA, at different concentration ratios. The recombinant HSV1-LacZ virus was identified by isolation of cells with a blue plaque phenotype after X-gal staining. To this purpose, cells were fixed with glutaraldehyde 1.5% in PBS, washed three times with PBS, and then incubated at 37° C. in the dark with 14 mM K₄Fe (CN)₆.3H20, 12 mM K₃Fe (CN)₆, X-gal (28.6 μl/ml, Sigma).

The tat cDNA (350 bp) was obtained from pCV-tat (60) following digestion with PstI and then ligated into the PstI site of plasmid pTZ18U (Sigma) to generate plasmidpTZ18U-Tat. The plasmid pB41-tat, containing the tat cDNA inserted in the UL41 locus of HSV1 under the control of the HSV-1 ICP0 pr, was obtained from pB41-lacZ by replacing lacZ coding sequences with tat cDNA. Briefly, tat cDNA was obtained from pTZ18U-Tat following digestion with HindIII-blunted/Xba, and inserted into EcoRI-blunted/XbaI sites of pB41-lacZ plasmid (35, 59). Finally, the HSV1-ICP0 pr was substituted with the HCMV promoter derived from the commercial vector pcDNA3.1 (Invitrogen Life Technologies), following digestion with NruI/PmeI (both are blunt end sites) and insertion into Sinal site of pB41-tat plasmid to generate vector pB41-HCMVtat (FIG. 1). The recombinant live attenuated HSV1-Tat vector was constructed by means of homologous recombination between UL41 sequences of the pB41-tat plasmid and the HSV1-LacZ vector, using the previously described Pac-facilitated lacZ substitution method (35, 59). Briefly, Vero cells were co-transfected with the HSV1-LacZ viral DNA cleaved with Pad (in order to excise lac Z) and the pB41-tat plasmid DNA linearised with NotI, at different concentration ratios. The recombinant HSV1-Tat was then identified by isolation of cells with a clear plaque phenotype after X-gal staining, performed as described above.

The HSV1-LacZ and HSV1-Tat viruses were purified by three rounds of limiting dilution each, followed by Southern-Blot (SB) analysis in order to confirm the presence of the transgenes, lacZ or tat. The viral DNAs were isolated from infected cell lysates using 10 mM Tris-HCl (pH 8.0), 10 mM EDTA, 0.6% SDS and proteinase K 0.25 mg/ml, and phenol:chloroform:isoamyl alcohol (25:25:1) and chloroform:isoamyl alcohol (25:1) extraction procedures (61). Aliquots of viral DNA were digested overnight at 37° C. with XbaI, fractionated by 0.8% agarose gel electrophoresis, transferred to a Hybond-N+ nylon membrane (Amersham Pharmacia, UK), and hybridised with lacZ or tat DNA sequences using the ECL Direct Nucleic Acid Labelling and Detection Systems Kit (Amersham) in accordance with the manufacturer's instructions.

### Large Scale Virus Stock Purification

HSV1-Tat, HSV1-LacZ and wild-type HSV (HSV1 LV) stocks were prepared by infecting Vero cells (4×10⁸) with increasing doses [0.01 to 0.05 multiplicity of infection (m.o.i.)] of each virus in 15 ml of medium for 1 hour at 37° C. under mild agitation. The viral inoculum was then removed and cells cultured at 37° C. until a 100% cytopathic effect was evident. The cells were then collected by centrifugation at 2000 rpm for 15 minutes. Supernatants containing the released virus were spun at 20,000 rpm in a JA20 rotor (Beckman) for 30 minutes to collect the virus, whereas the cellular pellets were resuspended in 2 ml of medium, subjected to three cycles of freeze-thawing (−80° C./37° C.), and subsequently to a single burst of sonication to release the viral particles. The virus was further purified by density gradient centrifugation (Opti Prep; Life Technologies, Inc.) and resuspended in PBS without calcium and magnesium. Viral stocks were titred in vitro, according to standard procedures (61), and stored at 80° C. Titres of viral stocks ranged between 7×10⁹ to 2×10¹¹ plaque forming units (pfu)/ml.

### Western Blot Analysis

Tat protein expression from the recombinant vector was analysed in BALB/c or Vero cells (1×10⁶ cells) infected with HSV1-Tat at m.o.i. of 1. Cell extracts, corresponding to 10 μg of total proteins, were loaded onto 12% SDS-polyacrylamide gels and analysed by Western blot procedure using a rabbit anti-Tat polyclonal serum (Intracel) at 1:1000 dilution and a mouse anti-rabbit horse radish peroxidase (HRP)-conjugated secondary antibody (Sigma) at 1:4000 dilution Immunocomplexes were detected by means of the ECL Western Blot detection kit (Amersham, Pharmacia Biotech). Controls were represented by uninfected cells and cells infected with 1 m.o.i. of HSV-LacZ control vector. A recombinant Tat protein (1 μg), obtained from Diatheva (Urbino, Italy), was included in each gel as control.

### Peptides

Peptides were synthesised by solid phase method and purified by HPLC to >98% purity (UF Peptides, University of Ferrara). HSV1 K^{b}-restricted peptides SSIEFARL (SSI) (SEQ ID NO:3), derived from glycoprotein B (gB), ITAYGLVL (ITA) (SEQ ID NO:4), derived from glycoprotein K (gK), and QTFDFGRL (QTF) (SEQ ID NO:5), derived from ribonucleotide reductase 1 (RR1), were used to evaluate anti-HSV1 T cell responses in C57BL/6 mice; HSV-1 K^{d}-restricted peptide DYATLGVGV (DYA) (SEQ ID NO:6), derived from ICP27, and SLKMADPNRFRGKDLP (SLK) (SEQ ID NO:7), derived from glycoprotein D (gD), were used to evaluate anti-HSV1 T cell responses in BALB/c mice. Peptide stocks were prepared in DMSO at 10⁻² M concentration, stored at −20° C., and diluted in RPMI 1640 before use.

### In Vivo Titration of Wild-Type HSV1 and of Live Attenuated HSV1-Tat or HSV1-LacZ

Animal use was according to European and Institutional guidelines. The lethal dose (LD₁₀₀) of wild-type HSV1 was determined both in BALB/c and C57BL/6 female mice since the susceptibility to HSV1 infection varies depending on gender and strains of mice (43-45). To this purpose, 8-week old BALB/c and C57BL/6 female mice (Charles-River Laboratories Calco, Lecco, Italy) were pre-treated, one week before challenge, with 2 μg/100 μl of Depo-Provera® (Depo-medroxy-progesterone acetate; Pharmacia & Upjohn) subcutaneously in the neck, to bring the mice at the same oestrus stage and render them more susceptible to HSV infection (46). Mice (n=7) were inoculated intravaginally with a range of 1.5×10⁴ to 1.5×10⁸ pfu of wild-type HSV1 (strain LV), to determine the Lthoo for challenge experiments. Before injection, the virus was thawed on ice, sonicated for 5 seconds, and stored on ice.

Mice were anaesthetised with 5% isofluorane to allow scraping of the vagina with a pipe scraper (in order to remove the mucus that could trap the virus) and then inoculated with the purified virus (in 10 μl of total volume for each mouse) using a pipette-tip. After infection, mice were observed daily to monitor the appearance of local and/or systemic clinical signs of infection including death. Disease severity was measured using the following arbitrary scores: 0 (no signs of infection), 1 (appearance of ruffled hair), 2 (appearance of cold sores on and around the vagina), 3 (appearance of paralysis of the back limbs) and 4 (mouse death). Each titration experiment was repeated 3 times. BALB/c mice died at the dose of 1.5×10⁶ pfu whereas C57BL/6 mice at the dose of 1.5×10⁸. Accordingly, these doses were used as LD₁₀₀ for challenging mice treated with HSV1-LacZ or HSV1-Tat.

Similar titration experiments were performed in BALB/c mice to determine the attenuation of the HSV1-Tat and HSV1-LacZ vectors using doses ranging from 10⁴ to 10⁸ pfu/mice administered by intravaginal route. At the dose of 10⁸ pfu/mouse a 20% survival was still evident indicating the attenuation of the recombinant viruses since the wild-type HSV1 had a two log lower LD₁₀₀ in the same mice strain.

Mice Immunisation with Live Attenuated HSV1-Tat or HSV1-LacZ and Challenge with Wild Type HSV1

Six-week-old C57BL/6 and BALB/c female mice (n=7) were treated intravaginally with 10² to 10⁴ pfu/mouse (10 μl) of the recombinant, replication-competent HSV1-Tat virus, or with the control virus HSV1-LacZ, as described in the previous section, to determine the optimal dose of virus for immunisation. Fifteen days after inoculation, mice were sacrificed to analyse anti-HSV1 immune responses. The dose of 10³ pfu/mouse was chosen as the optimal immunisation dose since it did not induce appearance of signs of infection and induced immune responses. For immunisation experiments, six-week-old C57BL/6 and BALB/c female were treated intravaginally with 10³ of HSV1-Tat and with HSV1-LacZ. Each experimental group was composed of 15 animals. Seven days post-infection mice (n=5/experimental group) were sacrificed to evaluate on fresh splenocyte cultures (individual mice), the presence of HSV1-specific T cell responses by means of IFN-γ and IL4 Elispot assays. In some experiments, at day 20, the presence of HSV1-specific IgM, IgG and IgA in sera, and of IgG and IgA in vaginal washes, was evaluated by enzyme-linked immunoassays (ELISA) in 5 mice/experimental group. At day 28, mice (n=10/experimental group) were challenged intravaginally with a lethal dose of wild-type HSV1 LV strain (10⁶pfu for BALB/c and 10⁸pfu for C57BL/6). After challenge, mice were observed daily to monitor their health, and the appearance of clinical signs.

Disease severity was measured using the following arbitrary scores as described in the previous paragraph. Each experiment was repeated 3 times. Seven days before immunisation and challenge all animals were treated subcutaneously with Depo-Provera® as described above. At sacrifice, mice were anesthetised intraperitoneally with 100 μl of isotonic solution containing 1 mg of Zoletil (Virbac, Milan, Italy) and 200 μg Rompun (Bayer, Milan, Italy) to collect blood, vaginal lavages and spleens. In some experiments C57BL/6 mice were also immunised intradermally with 10³ pfu in a volume of 100 μl.

### Collection of Samples

Blood samples were incubated for 16 hours at 4° C., centrifuged for 10 minutes to obtain sera and stored at −80° C. until analysis. Samples of vaginal secretions were obtained by washing the vaginal cavity with 250 μl of PBS containing 5% of FBS and a cocktail of protease inhibitors (Roche Diagnostics, Mannheim-Germany) and then incubated on ice for 1 hour. Samples were centrifuged at 10,000 rpm in an Eppendorf microfuge for 10 minutes at 4° C., treated with 5 μl of 1M PMSF and 5 μl of 1% NaN₃, and then stored at −20° C. until analysis.

Splenocytes were purified from spleens squeezed on filters (Cell Strainer, 70 μm, Nylon, Becton Dickinson). Following red blood cell (RBC) lysis with RBC lysing buffer (Sigma), cells were washed with RPMI 1640 (Cambrex) containing 10% FBS (Hyclone), spun for 10 minutes at 1000 rpm in a bench centrifuge, resuspended in RPMI 1640 containing 10% FBS, 1% L-glutamine (BioWhittaker, Walkersville, Md.), 1% penicillin/streptomycine (BioWhittaker, Walkersville, Md.), 1% nonessential aminoacids (Sigma), 1 mM sodium piruvate (Sigma) and 50 mM beta-mercaptoethanol (Gibco, Grand Island).

### Elispot Assays

IFN-γ or IL4 Elispot assays were carried out using the murine kits provided by Becton Dickinson, according to the manufacturer's instructions. Briefly, nitrocellulose plates were coated with 5 μg/ml of anti-IFN-γ or anti-IL4 mAb for 16 hours at 4° C. Plates were then washed with PBS and blocked with RPMI 1640 supplemented with 10% FBS for 2 hours at 37° C. Total splenocytes from individual mice (5×10⁵ cells) were added to the wells in duplicate and incubated with HSV1 derived peptides (10⁻⁶M) for 24 hours at 37° C. Controls were represented by cells incubated with 5 μg/ml of Concanavaline A (positive control) or with medium alone (negative control). Spots were quantified using an AELVIS 4-Plate Elispot Reader (TEMA ricerca s.r.l. Bologna, Italy). The number of spots counted in the peptide-treated cultures minus the number of spots counted in the untreated cultures was the specific response.

Results are expressed as number of spot forming units (SFU)/10⁶ cells. Values at least 2-fold higher than the mean number of spots in the control wells (untreated cells) and ≧50 SFU/10⁶ cells, were considered positive.

### Analysis of Antibody Responses

Anti-HSV1 specific antibodies in sera (IgM, IgG, IgG1 and IgG2a titres) were measured on samples collected from individual mice by enzyme-linked immunosorbent assay (ELISA) using 96-well immunoplates (Nunc Max Sorp) previously coated with 100 ng/well of HSV1 viral lysate (Herpes Simplex Type 1 Purified Viral Lysate, Tebu-bio), resuspended in PBS containing 0.05% NaN₃, for 16 hours at 4° C. Plates were washed five times with PBS (pH 7.0) containing 0.05% Tween 20 (Sigma) (washing buffer) using an automatic washer (BioRad Model 1575 ImmunoWash) and then blocked for 90 minutes at 37° C. by the addition of 200 μl/well of PBS containing 0.5% milk and 0.05% NaN₃.

After extensive washes, 100 μl/well of appropriate dilutions of each serum were dispensed in duplicate wells and then incubated for 90 minutes at 37° C. Plates were washed again before the addition of 100 μl/well of HRP-conjugated goat anti-mouse IgG (Sigma), diluted 1:1000, or HRP-conjugated goat anti-mouse IgM (Sigma), diluted 1:7500, in PBS containing 0.05% Tween 20 and 1% BSA, and incubated at 37° C. for 90 minutes. In each plate, two wells were incubated with PBS containing 0.5% milk and 0.05% NaN₃ and the secondary antibodies (blank). Analysis of anti-HSV1 IgG isotype was determined using a goat anti-mouse antibody directed against IgG1 or IgG2a (Sigma), diluted 1:30,000 in PBS containing 0.05% Tween 20 and 1% BSA. After incubation, plates were washed five times and subsequently a solution of 2,2′-Azinobis[3-ethylbenzothiazoline-6-sulfonic acid]-diammonium salt (ABTS) substrate (Roche) was added. The reaction was stopped with 0.1M citric acid after 50 minutes. The absorbance values were measured at 405 nm with an automatic plate reader (SUNRISE TECAN Salzburg-Austria). The cut-off value was estimated as the mean OD of 3 negative control sera plus 0.05. Each OD value was subtracted of the blank and cut-off values to obtain a net OD value. IgG titres were calculated by intercept function using the Excel program.

For analysis of IgA, Maxisorp 96-well plates were coated for 1 hour at 37° C. with 0.1 ml of 100 ng/well of HSV1 viral lysate (in PBS containing 0.05% NaN₃) to measure specific IgA, and with 0.1 ml of a goat anti-mouse IgA serum (Sigma) (0.2 mg/ml in PBS containing 0.05% NaN₃) to measure total IgA and to generate a standard curve. Plates were washed six times with washing buffer (0.3 ml/well), saturated with PBS containing 1% BSA and 0.1% Tween 20 (0.2 ml/well), and incubated for 1 hour at 37° C. Plates were drained, and samples tested (duplicate wells) starting from 1:20 dilution for total IgA (0.1 ml/well), and at 1:8 dilution for antigen-specific IgA (0.1 ml/well). For the standard curve, mouse IgA (Sigma) were used from 1.5 ng/ml to 100 ng/ml. After 1 hour at 37° C., wells were washed six times, incubated with a goat biotin-conjugated anti-mouse IgA serum (Sigma) (0.1 ml/well) for 1 hour at 37° C., washed and incubated with HRP-conjugated streptavidin (BD-Pharmingen San Diego, Calif.) (0.1 ml/well) for an additional 30 minutes at 37° C. ABTS substrate was added and colour development measured after 10 minutes incubation at 37° C., as described above. All plates included positive and negative control samples. The amount of antigen-specific IgA was expressed as percentage (%) of specific IgA with respect to the total amount of IgA in the same sample.

### Results

### Construction of a Live Attenuated HSV1 Vectors

Two live attenuated HSV1 vectors (FIG. 1), containing the lacZ gene (HSV1-LacZ) or the HIV-1 tat gene (HSV1-Tat) in the UL41 non-essential locus of the HSV1 genome (LV strain), were constructed by means of a two-step method (59). First, the HSV1-LacZ virus was generated by homologous recombination between wild-type HSV1 and the pB41-lacZ plasmid containing the lacZ marker gene under the control of the ICP0 immediate early promoter of HSV inserted in the UL41 gene of HSV1. Next, the HSV1-Tat recombinant virus was generated by homologous recombination between the pB41-HCMVtat plasmid and the viral HSV-LacZ DNA. The presence of lacZ and tat genes in the UL41 locus of both recombinant viruses was confirmed by Southern blot analysis (data not shown).

The expression of Tat protein was determined by Western blot analysis. To this purpose, BALB/c fibroblast cell lines were infected with HSV1-Tat and Tat expression was analysed after 12 and 24 hours post-infection. Uninfected cells and cells infected with wild-type HSV1 (LV strain) and HSV-LacZ viruses represented the negative controls, whilst recombinant Tat protein acted as the positive control. As shown in the FIG. 1C, Tat expression was detected at 12 hours post-infection. Similar results were observed in Vero cells (data not shown).

Analysis of HSV1-Specific T Cell Responses in Mice Infected with Recombinant HSV1 Vectors

To determine the effect of the presence of Tat in HSV1 recombinant vectors on the induction of HSV1-specific T cell mediated responses, C57BL/6 and BALB/c female mice were infected intravaginally with 10³ pfu of the attenuated replication-competent HSV1-LacZ or HSV1-Tat recombinant viruses. After 7 days, the presence of HSV1-specific T cell responses was evaluated by IFN-γ and IL4 Elispot assays on fresh splenocytes. To this end, T cell responses in C57BL/6 mice were evaluated using three K^{b}-restricted CTL peptide epitopes, including the immunodominant SSIEFARL (SSI) (SEQ ID NO:3) and ITAYGLVL (ITA) (SEQ ID NO:4) epitopes, respectively derived from HSV1 glycoprotein B and glycoprotein K, and the subdominant QTFDFGRL (QTF) (SEQ ID NO:5) epitope derived from ribonucleotide reductase 1. Similarly, T cell responses in BALB/c mice were evaluated using two peptides, including the peptide SLKMADPNRFRGKDLP (SLK) (SEQ ID NO:7) containing K^{d}-restricted CD4 and CD8 immunodominant epitopes derived from glycoprotein D, and the CTL subdominant epitope DYATLGVGV (DYA) (SEQ ID NO: 6) derived from ICP27.

As shown in FIG. 2, splenocytes from C57BL/6 mice infected with HSV1-LacZ released IFN-γ in response to the immunodominant SSI and ITA CTL epitopes, but not to the subdominant QTF epitope. In contrast, mice infected with HSV1-Tat responded to all three epitopes including the subdominant QTF epitope, and, remarkably, responses to the immunodominant SSI and ITA peptides were significantly higher than those developed in control mice. Th2-type responses, as measured by IL4 release, were absent in both groups of mice. These results unexpectedly demonstrate that the presence of Tat in an attenuated HSV1 vector increases and broadens Th1-type immune responses directed to HSV1 in C57BL/6 mice.

Since fresh splenocytes from BALB/c mice poorly responded to HSV1 peptide epitopes (data not shown), splenocytes were re-stimulated in vitro for 5 days with the peptides and then assayed by IFN-γ and IL4 Elispot. As shown in FIG. 3, splenocytes from mice infected with HSV1-LacZ released IFN-γ and IL4 in response to SLK and DYA peptides, while mice infected with HSV1-Tat released IFN-γ but not IL4 in response to the SLK and DYA peptides.

Remarkably, the response to the subdominant DYA peptide was significantly higher in mice treated with HSV1-Tat.

Similar results were obtained when C57BL/6 mice were infected intradermally with the two HSV1-derived vectors (FIG. 4).

In conclusion, these results demonstrate that the intravaginal or intradermal infection of two different strains of mice with a recombinant HSV1 vector expressing Tat induces stronger HSV1-specific cellular responses compared to the infection with a recombinant HSV1 control vector.

Analysis of HSV1-Specific Humoral Response in Mice Infected with Recombinant HSV1 Vectors

To evaluate the capacity of HSV1 recombinant vectors to induce antibodies against HSV1, sera and vaginal washes from C57BL/6 and BALB/c mice were collected at day 20 after intravaginal or intradermal infection with HSV1-Tat or HSV1-LacZ and evaluated for the presence of anti-HSV1 IgM, IgG, and IgA using ELISA assays. As shown in FIG. 5, blood HSV1-specific IgG titres were detected in a few mice infected intravaginally with HSV1-Tat, but never in mice infected with HSV1-LacZ, nor in mice infected intradermally with HSV1-Tat or HSV1-LacZ (data not shown). Surprisingly, independently of the mouse strain, the intravaginal infection with HSV1 vectors expressing Tat induces the generation of HSV1-specific humoral responses. Furthermore, the IgG isotype was analysed and the reported results demonstrate the presence of IgG2a but not of IgG1 antibodies, indicating a Th1-type immune response (FIG. 5). We did not detect the presence of IgA and IgM in the sera as well as of IgG and IgA in vaginal washes in mice infected intravaginally or intradermally with the two HSV1-derived vectors (data not shown).

Analysis of Protection in Mice Treated with Attenuated HSV1 Recombinant Vectors Against Wild-Type HSV1 Challenge

Altogether, the evaluation of immune responses in C57BL/6 and BALB/c mice infected with HSV1-Tat shows that an attenuated HSV1-derived vector expressing Tat induces higher and broader anti-HSV1 cellular and humoral responses of the Th1-type in both strains of mice, compared to those developed in mice treated with the HSV1-LacZ vector. To determine whether the increased and broader immune responses confer greater protection from HSV1 challenges, groups of BALB/c and C57BL/6 mice were treated intravaginally with 10³ pfu of HSV1-Tat or HSV1-LacZ and challenged at day 28 with a lethal dose of wild-type HSV1. Disease severity was scored on a scale of 0 (no signs of disease) to 4 (mouse death) as specified in Materials and Methods. As represented in FIG. 6, all C57BL/6 and BALB/c mice treated intravaginally with HSV1-LacZ progressively developed severe HSV1 disease and died, while mice treated with HSV1-Tat presented a mild and transient disease and all of them survived to the viral challenge. Similar experiments were performed in C57BL/6 mice treated intradermally with 10³ pfu of HSV1-Tat or HSV1-LacZ and challenged at day 28 with a lethal dose of wild-type HSV1. As represented in FIG. 7, C57BL/6 mice treated with HSV1-LacZ developed a mild and transient disease while mice treated with the HSV1 vector expressing Tat did not show any disease sign protecting mice from HSV1 lethal challenge.

The above results demonstrate that HIV1 Tat, when expressed by a replication-competent HSV1 vector, not only does not dysregulate the immune system against HSV infection, but increases and broadens the Th1-like and CTL responses against HSV1 immunodominant and subdominant T cell epitopes and, in addition, elicits detectable IgG responses. A similar attenuated HSV1 recombinant vector without Tat (HSV1-LacZ) induces lower T cell responses, which are directed only against the immunodominant epitopes, and generates no measurable IgG serum responses. Protection is so good that immunisation of BALB/c and C57BL/6 mice with HSV1-Tat protected the mice against HSV lesions and death after challenge with a lethal dose of wild type HSV1 virus. No such protection was observed when HSV1-LacZ was used.

### Example 2

The replication-defective HSV1-ΔICP4-ΔUL41-ΔgJ (derived from HSV1 strain KOS) virus has a deletion in ICP4, in UL41 and in the gene encoding for the envelope glycoprotein J. Tat cDNA is inserted in ΔgJ.

Replication-defective recombinant viruses are generated from the S0ZgJHE and T0ZGFP backbone vectors which are deleted respectively: (i) in one out of five immediate early (IE) genes (ICP4−), in the glycoprotein gJ containing GFP and in the sequence of UL41 locus containing the LacZ gene; (ii) in three IE genes (ICP4⁻/ICP27⁻/ICP22⁻GFP) and in the sequence of UL41 locus containing LacZ. The Tat gene replaces the GFP gene and LacZ respectively in S0ZgJHE and T0ZGFP backbone vectors. The resulting vectors were designated S0ZgJHTat and T0TatGFP.

The T0TatGFP have been described in our paper (Bozac A. et al Vaccine 1996). The in vivo experiments are on going

### Generation of Plasmids and Recombinant Replication-Defective HSV-1 T0TatGFP Vector

Plasmid pCV-tat, expressing the HIV-1 tat cDNA (HTLV-IIIB isolate, subtype B) has been previously described [Aldovini, A. et al. Proc Natl Acad Sci USA 1986]. Plasmid DNA was purified from Escherichia coli using the Qiagen endotoxin-free Maxi Kit (Qiagen, Hilden, Germany). Plasmid pB410-tat was constructed by introduction of the HIV-1 tat cDNA (350 bp) from pCV-tat into the UL41 locus of plasmid HSV-1 pB41 that has been described elsewhere [Krisky et al. Gene Ther. 1998]. The tat cDNA, under the transcriptional control of the HSV immediate-early ICP0 promoter, was inserted into EcoRI/XbaI sites of pB41 plasmid between the two UL41 HSV fragments (HSV genomic positions 90145-91631 and 92230-93854) [Krisky et al. Gene Ther. 1997]. The T0Z-GFP is a replication-defective HSV-1 viral vector characterized by low cytotoxicity due to the deletion of three immediate early genes (ICP4, ICP27, which are essential for viral replication and ICP22 which is not) and contains the gfp gene in the ICP22 locus and also the lacZ gene in the UL41 locus as marker genes. Plasmid pB410-tat was constructed to genetically recombine with the genome of the T0Z-GFP viral vector using the previously described Pac-facilitated lacZ substitution method [Krisky et al. Gene Ther. 1997, Fraefel C. et al. Methods Mol Biol. 2011]. The generation of recombinant viruses was carried out using the standard calcium phosphate transfection procedure with 5 μg of T0Z-GFP viral DNA and 1 μg of linearized plasmid pB410-tat. Transfection and isolation of the recombinant viral progeny was performed in Vero-ICP4 and ICP27 stably-transfected 7b cells, as previously described [Marconi, P. et al Proc Natl Acad Sci USA 1996, Krisky, D. et al. in Gene Therapy Protocols 1997].

The recombinant T0-tat virus containing the tat cDNA was first identified by isolation of a clear plaque phenotype after X-gal staining. The T0-tat virus was purified by three rounds of limiting dilution technique and the presence of the transgene was confirmed by Southern blot analysis. Viral stocks of the T0-tat and of the control vector T0-GFP (derived from T0Z-GFP without lacZ reporter gene in UL41 locus) were prepared and titered using 7b cells.

Preliminary studies with replication-defective recombinant T0TatGFP are carried out to test the capacity of Tat to broaden the immune responses against HSV after intradermal immunization (i.d.). BalbC mice were immunized with 1×10⁷ pfu of T0TatGFP and T0ZGFP viruses or PBS-A 1×. The above-described doses of recombinant virus were administered in 100 μl for the i.d. route. Animals were boosted with the same dose of virus i.d. at weeks 3 and 7, or at weeks 3 and 12 after priming. The animals are challenged with HSV-1 and HSV-2 wild type viruses to confirm that, in the groups of mice immunized with the vector expressing Tat, T0TatGFP increases cross-protection responses against HSV1 and 2, has an increased rate of survival and protection against both HSV-1 and HSV-2 wild types.

### Example 3

Replication-competent HSV1-434.5-ΔUL41-ΔgJHE-AIGR20 (derived from HSV1 strain F) is an highly attenuated virus. The reference viral vector, HSV-R316, that was used for the construction of the attenuated vector backbone, has a deletion in both copies of the γ34.5 gene, the major neurovirulence determinant of HSV-1 (62-64). Into this vector backbone were introduced three expression cassettes, respectively, in two virulence genes non-essential for virus replication, and in one intergenic non-coding region IGR20. This mutant virus carries LacZ gene in UL41 locus (HSV genomic positions 90145-91631 and 92230-93854), EGFP gene in the Us5 locus corresponding to the glycoprotein gJ (HSV-1 map position 137626-137729) and Luciferase gene into the non-coding HSV-1 intergenic region 20 (IGR20), between UL26.5 and UL27 HSV-1 sequences (HSV-1 map position 52878-52910). Insertion of luciferase (Luc) marker gene in the IGR20 region allows biodistribution studies in vivo.

### Example 4

The replication-defective HSV1-ΔICP27-IGR20Luc-ΔgJ (derived from HSV1 strain F) virus (named HSVLucΔ27gJTat) has a deletion in the ICP27 gene essential for viral replication, in IGR20 (intergenic non coding region 20) containing the luciferase gene, and in the gene encoding for the envelope glycoprotein J (ΔgJ) where Tat cDNA is inserted.

The idea is that a non-replicative HSVLucΔ27gJTat vector can promote signals favouring the emergence of a Th1 immune response not only against dominant epitopes but also against subdominant epitopes of the IE proteins ICP4 of HSV, that seems to be crucial to halt viral infection. In fact, Posavad and co-workers [65] have demonstrated that seronegative patients (IS) without serum Abs to HSV-1 or HSV-2 and no clinical or virological evidence of mucosal HSV infection possessed consistently detectable HSV-specific T cell responses against some viral proteins such as UL39 and the IE proteins ICP4 and ICP0. Therefore, the antigenic repertoire of T cells in IS subjects is skewed compared with that of HSV-2(+) subjects, suggesting that IS subjects had more frequent T cell responses to IE proteins and infrequent T cell responses to virion components. Based on these observations we have developed a non-replicative vector (HSVLucΔ27gJTat) deleted in ICP27 IE but with wild-type ICP4 gene for vaccine strategies against HSV infection.

The construction of this vector is described below.

The plasmids pTZgJHE and pgJ-Tat were first generated in order to create the new vectors required for generation of the non-replicative vector Δ27gJTat.

The EGFP and luciferase cytokine transgenes were derived from commercial plasmids pIRES-EGFP (Clontech), pGL3Luc (Promega) respectively. All the DNA fragments were excised with New England Biolabs (NEB) enzymes and purified from agarose gel with Millipore Kit (LSKGEL050) after electrophoresis run.

All the fragments obtained by PCR were done by amplify the regions of interest with DNA polymerase High-Fidelity (Phusion Hot Start, FINNZYMES)

Construction of pgJ1, pgJHE and pgJTat Plasmids

The 2036 by of SalI-HindIII fragment from the HSV-1 genome (nucleotides 136308 to 138345) containing Us5 locus, which encodes for a non-essential glycoprotein J, was cloned into pTZ18U plasmid. The resulting plasmid, pTZgJSalI-HindIII was used to generate pTZgJHE, containing a deletion in US5 between the TATA box and the gJ coding sequence (nucleotides 137626-137729), by insertion of the EGFP coding sequence, derived from NheI/XhoI digestion of plasmid pcDNA3.1Hyg(+)EGFP, driven by the cytomegalovirus (CMV) promoter into the SphI (137626) and NruI (137729) sites of pTZgJSalIHindIII.

To construct pgJTat, the Tat sequence was obtained from pCV-tat plasmid, expressing the HIV-1 tat cDNA (HTLV-IIIB isolate, subtype B) previously described (Aldovini, A. Proc Natl Acad Sci USA 1986). As a first step, Tat was cloned into HindIII-Xba of a pCDNA 3.1 Hygro+ plasmid and, as a second step, HCMV-tat expression cassette was cut and cloned in NruI-SphI of pTZ18UgJ1 (named pgJ1), which contains the sequence of HSV-1 US5 locus (gJ) (genomic position: 136308-138345).

### Recombinant Vector Construction by BAC “Recombineering” Method in Bacteria Cells

The cloning of a large DNA virus genome, such as that of HSV-1 as bacterial artificial chromosome (BAC), has facilitated the easy construction of recombinant viruses by homologous recombination in Escherichia coli [66].

A HSV-1 (strain F) genome cloned into a BAC, flanked by loxP sites, was maintained in SW102 bacteria, a AgalK specific strain [66, 67]. The BAC-HSV genome was modified by a galK-based selection in these specific bacteria. Primers were designed to have 20 by of the 5′ and 3′ galK expression cassette plus 50 by of HSV homology sequence, situated at the locus where the deletion or the insertion is desired. Subsequently the galK expression cassette, flanked by the 50 by of homologous HSV sequences, was amplified by PCR.

The obtained PCR fragment was inserted into the HSV-genome by electroporation in SW102 bacteria [67] and the galK positive colonies were evaluated by screening on MacConkey-galactose added plates; only the bacteria that had acquired the fragment were able to grow on the medium.

In the second step the galK gene was substituted with a specific gene expression cassette of interest with the same 50 by of HSV homology sequences used for the galK insertion. As done previously, the gene expression cassette of interest was amplified by PCR reaction and, subsequently, was inserted by electroporation into bacteria. To evaluate the substitution between the galK gene and the gene of interest, the galK negative colonies were grown on 2-deoxy-galactose medium with added glycerol (DOG). The SW102 bacteria were grown in LB medium, the DNA was extracted and purified with a QIAgen Miniprep kit (QIAGEN®), and screened with appropriate restriction enzyme digestion.

In this last step, the BAC was deleted from the HSV genome by co-transfection with HSV-BAC DNA and a Cre-Recombinase expression plasmid on Vero cells. To evaluate the excision of the BAC, the samples were analyzed by Southern blot and specific PCR reaction, while the presence of the gene of interest was evaluated by Southern blot, Western blot and biological activity.

Cell Lines and Culture Conditions

Vero (ATCC, Rockville, Md.) and Vero 2.2 cells, (that are stably transfected with HSV-1 ICP27) were maintained in high glucose Dulbecco's modified Eagle's medium D-MEM (EuroClone) supplemented with 2 mM L-Glutamine, 100 units/ml Penicillin, 100 μg/ml Streptomycin and 10% Fetal Bovine Serum (USA approved, EuroClone). Vero 2.2 cells were subjected monthly to selection with 1 mg/ml of Geneticin (G418, Roche).

### Recombinant Bac Vectors Construction

BAC HSV-1-IGR20Luc Vector (BAC-HSVLuc Vector)

The BAC-HSVLuc vector, was generated from a pre-existing BAC-cloned HSV-1 strain F genome [66] (kindly given by Cornel Fraefel) containing the BAC (bacterial artificial chromosome) vector inserted into the intergenic region between U_{L}3 and U_{L}4 of a full-length infectious HSV-1 DNA [66]. The BAC-HSVLuc recombinant vector was done in bacteria, using the “recombineering” system described above. In this way the PCR fragments, expressing galK as a first step, and then, as second step, substituted with a pHCMV-Luciferase-BGHpolyA cassette, were introduced by homologous recombination into E. coli using the galK positive and negative selection procedure into the non-coding HSV-1 intergenic region 20 (IGR20), between U_{L}26.5 and U_{L}27 HSV-1 sequences (HSV-1 map position 52878-52910) (FIG. 8A).

BAC HSV-1-IGR20LucΔICP27 Vector (BAC-HSVLucΔ27 Vector):

the BAC-HSVLucΔICP27 recombinant vector was constructed by using the “recombineering” system [67]. BAC-HSVLucΔ27 vector was constructed through the complete deletion of the ICP27 coding sequence starting from the BAC-HSVLuc genome. ICP27 is an immediate-early protein essential for viral replication that regulates the synthesis of early (E) and late (L) proteins. Deletion of this gene leads to an abortive infection with no yield of progeny virus.

To generate a recombinant defective vector having a deleted ICP27 gene, primers were designed to have 20 by of the 5′ and 3′ galK expression cassette plus 50 by of HSV ICP27 homology sequences. In this way, all of the ICP27 coding sequence (HSV map position 113497-115227) was deleted. BAC-HSVLucΔ27 DNA was isolated from several colonies and characterized by restriction enzyme analysis.

The recombinant virus HSVLucΔ27 (FIG. 8B) was finally obtained following transfection of the BACLucΔ27 DNA with a Cre-Recombinase expression plasmid into Vero2.2 cells (complementing in trans the ICP27 protein).

Infections on different cell lines proved that HSV-LucΔ27 virus was able to grow only in the complementing cell line, confirming that HSV-LucΔ27 is a replication-defective virus and that ICP27 expression is essential for its growth.

HSV-1-IGR20LucΔICP27gJHE (HSVLucΔ27gJHE)

The HSVLucΔ27gJHE vector (FIG. 8C) was then obtained by homologous recombination in Vero 2.2 cells [68], by co-transfecting 5 μg of HSVLucΔ27 DNA and 1 μg of the pTZgJHE recombinant plasmid described above, where the pHCMV-EGFP cassette was inserted into the HSV-1 Us5 region, between the TATA box and the coding sequence of glycoprotein J (HSV-1 map position 137626-137729). The virus was purified by three rounds of limiting dilutions each followed by Southern blot analysis in order to confirm the deletion and the presence of the transgene [69].

HSV-1-IGR20LucΔICP27gJTAT (HSVLucΔ27gJTat Vector)

HSVLucΔ27gJTat vector (FIG. 8D) was obtained by homologous recombination, using standard calcium phosphate transfection, of 5 μg of HSVLucΔ27gJHE viral DNA and 1 μg of linear plasmids pgJ1tat carrying HIV-1 Tat flanked by gJ HSV viral sequences. Transfection and isolation of the recombinant virus was performed in Vero2.2 cells (modified Vero cells) capable of providing the essential ICP27 HSV gene product. The recombinant virus (HSVLucΔ27gJTat) containing the tat cDNAs was identified by isolation of a clear plaque phenotype for GFP under the fluorescent microscope. The virus was purified by three rounds of limiting dilutions each followed by Southern blot analysis in order to confirm the deletion and the presence of the transgene.

### Example 5

The replication-defective HSV1-ΔICP4-ΔUL41tB5Ag-ΔgJTat (name SHtB5Ag/gJT at) (derived from HSV1 strain KOS) virus has a deletion in ICP4, in UL41 and in the gene encoding for the envelope glycoprotein J. This vector contains Mycobacterium tuberculosis (Mt) cDNA antigens (tB5Ag) in Ul41 and Tat cDNA in ΔgJ respectively. tB5Ag sequence (Delogu et al. Infect Immun 2000; Delogu et al. Infect Immun 2002) expresses a fusion protein that includes several mycobacterium antigens (Ag85B, ESAT-6, Mpt64, Mpt63, Mpt83), a TPA (tissue plasminogen activator signal sequence), which ensures the release of the fusion protein, and a sequence codifying for HA epitope (haemagglutinin) that allows the identification of the protein with a specific antibody therefor.

Replication-defective recombinant viruses SHtB5Ag/gJHE and SHtB5Ag/gJTat are generated respectively from the S0ZgJHE and S0ZgJHTat backbone vectors (Example 2) which are deleted: in one out of five immediate early (IE) genes (ICP4−), in the glycoprotein gJ containing GFP or Tat and in the sequence of UL41 locus containing the LacZ gene.

### Viral Vector Co-Expressing Tat as an Immunomodulatory Molecule and Mt Antigens as a Vaccine Strategy Against Mycobacterium tuberculosis

We investigated whether HIV-1 Tat is capable of inducing broad and protective immunity against TB.

### Material and Methods

### Cell Lines

Vero cells, a monkey kidney fibroblast cell line, Balb/c cells, a fibroblast cell line derived from Balb/c mice were grown in DMEM (Euroclone, Grand Island, N.Y.) supplemented with 10% FBS (Euroclone), 2 mM L-glutamine, 100 mg/ml penicillin and 100 U/ml streptomycin at 37° C. in 5% of CO₂ incubator. Human HeLa3T1 cells were grown in DMEM and 10% FBS; these cells contain an integrated copy of plasmid HIV-LTR-CAT where expression of the chloramphenicol acetyl transferase (CAT) reporter gene is driven by the HIV-1 LTR promoter and occurs only in the presence of biologically active Tat protein.

### Bacteria

Escherichia coli (Stratagene) strain DH5α was used in plasmid cloning procedures. Bacteria were grown in Luria-Bertani medium (for liquid culture) or in Luria-Bertani agar plates, both supplemented with antibiotics as appropriate (Ampicillin 100 μg/ml or Kanamycin 50 μg/ml).

### Generation of Plasmids and Recombinant Replication-Defective and Replication-Competent HSV-1 Vectors.

Plasmid pCV-tat, expressing the HIV-1 tat cDNA (HTLV-IIIB isolate, subtype B) has been previously described {Aldovini, A. Proc Natl Acad Sci USA 1986}. Plasmids pB410-tat and pgJ-tat were constructed by introduction of the HIV-1 tat cDNA (350 bp) from pCV-tat into the HSV-1 Us5 locus of plasmid pB41, described elsewhere {Krisky D. et al Gene Ther 1998} and into HSV-1 Us5 locus (gJ) of plasmid pSPgJ. The pB41tB5Ag plasmid was constructed by introduction of TB5Ag in SpeI/XbaI from pcDNA3.1/Hygro+ TB5Ag cassette {Delogu G. et al., 2001}, which contains a multigene under HCMV promoter encoding for 5 Mt antigens: Ag85B, ESAT-6, mpt 64/63/83 (tB5Ag), into pB41 plasmid between the two UL41 HSV fragments (HSV genomic positions 90145-91631 and 92230-93854) {Krisky et al. Gene Ther 1997} (FIGS. 1 and 2).

FIGS. 9 and 10 show the schematic representation of pcDNA3.1/Hygro+ TB5Ag and pB41tB5Ag plasmids, respectively.

S0Z-gJGFP is a replication-defective HSV-1 viral vector characterized by low cytotoxicity due to the deletion of an immediate early gene (ICP4) which is essential for viral replication, and contains the gfp gene in the Us5 locus (glycoprotein gJ), which codes for a non-essential glycoprotein, and the lacZ gene in the UL41 locus, as marker genes. Plasmid pBTB5Ag, pB410-tat and pgJ-tat were constructed to genetically recombine with the genome of the S0Z-gJHE viral vector using the previously described Pac-facilitated lacZ/GFP substitution method {Krisky et al. Gene Ther 1997}.

Generation of recombinant viruses was carried out using the standard calcium phosphate transfection procedure with 5 mg of S0Z-gJGFP viral DNA and 1 mg of linearized plasmids pB41TB5Ag or pB410-tat or pgJ-tat. Transfection and isolation of the recombinant viral progenies were performed in E5 cells, which are Vero cells stably transfected with HSV-1 ICP4 gene. The recombinants SHtBSAg/gJHE and SHtB5Ag/gJTat viruses containing the tBSAg or tat cDNA were first identified by isolation of a clear plaque phenotype after X-gal staining or GFP screening. The viruses were purified by three rounds of limiting dilution technique and the presence of the transgenes were confirmed by Southern blot analysis. Viral stocks of the SHtBSAg/gJHE, SHtBSAg/gJtat and of the control vector S0ZgJHE were prepared and titrated using E5 cells.

Protein expression was evaluated by western blot with specific monoclonal antibodies and Tat biological activity by CAT-ELISA technique.

### Western Blot Analysis

TB5Ag protein expression from the recombinant vector was analyzed in Vero cell line (1×10⁶ cells) and CB1 mouse dendritic cell line infected with SHtB5Ag/gJHE and SHtB5Ag/gJTat at multiplicity of infection (m.o.i.) of 1. Cell extracts, corresponding to 10 mg of total proteins, were loaded onto 10% SDS-polyacrylamide gel and analyzed by Western blot procedure using a mouse monoclonal anti-HA (Clone HA-7, SIGMA) at 1:1000 dilution and the anti-mouse IgG peroxidase conjugated secondary antibody (Pierce) at 1:2500 dilution Immunocomplexes were detected by ECL Western Blot detection kit (Amersham, Pharmacia Biotech). Controls were represented by uninfected cells and cells infected with 1 m.o.i of HSV-LacZ (S0Z) control vector.

### CAT ELISA Assay

To analyse the expression of functional Tat protein, HeLa3T1 cells (1×10⁶) were infected with replication-defective SHtB5Ag/gJTat and replication competent HSV-Tat and control vectors SHtB5Ag/gJHE and S0ZgJHE at two different m.o.i. (from 0.1 and 1) for 1 hour at 37° C. under mild shaking. After infection, cells were washed twice with complete medium to eliminate the virus particles that did not infect the cells, plated onto 6 well plates, and cultured at 37° C., 5% CO₂ for 6-12 and 24 hours. After incubation, cells were disrupted with lysis buffer and CAT expression was measured on cleared supernatants through the use of ELISA (enzyme-linked immuno-sorbent assay) microplates coated with anti-CAT antibody according to the manufacturer's instructions (Roche CAT ELISA Kit).

### Virus Stock Purification

Recombinant replication-defective and replication-competent HSV-1 stocks were prepared by infecting 4×10⁸ 7 b complementing cells or Vero cells respectively with 0.05 m.o.i. of recombinant viruses in suspension in 15 ml of medium for 1 hour at 37° C. under mild agitation. The infected cells were cultured at 37° C., 5% CO₂ until a 100% cytopathic effect was evident. The cells were then collected and centrifuged at 2000 rpm for 15 minutes. The supernatants were spun at 20000 rpm in JA20 rotor (Beckman) for 30 minutes to collect the virus. The cellular pellets were resuspended in 2 ml of medium, subjected to three cycles of freeze-thawing (−80° C./37° C.) and a single burst of sonication, to release the viral particles. The virus was further purified by density gradient centrifugation (Opti Prep; Life Technologies, Inc.) and resuspended in PBS-A 1×. Viral stocks were titrated as previously described and stored at −80° C. Titers averaged between 2×10⁸ to 2×10⁹ plaque forming units (pfu)/ml.

### Peptides

In order to study the immunoresponses against MT antigens and HSV antigens in Balb/c mice we used peptides that were synthesized by solid phase method and purified by HPLC to >98% purity (UF Peptides, University of Ferrara).

TB synthetized peptides: VNYQNFAVT (SEQ ID NO: 8) derived from MPT 64, FAPTNAAFD (SEQ ID NO: 9) derived from MPT83, AYPITGKLG (SEQ ID NO: 10) derived from MPT63, SLTKLAAAW (SEQ ID NO:11) and AYQGVQQKW (SEQ ID NO:12) derived from ESAT6, YAGSLSALL (SEQ ID NO:13), FSRPGLPVE (SEQ ID NO:14) and FQDAYNAAG (SEQ ID NO:15) derived from FBPB were used to evaluate anti-TB T cell responses in Balb/C mice.

HSV-1 K^{d}-restricted peptide DYATLGVGV (DYA) (SEQ ID NO:6), derived from ICP27, and SLKMADPNRFRGKDLP (SLK) (SEQ ID NO:7), derived from glycoprotein D (gD), were used to evaluate anti-HSV T cell responses in BALB/C mice. Peptide stocks were prepared in DMSO at 10⁻² M concentration, stored at −20° C., and diluted in RPMI 1640 before use.

Mice Immunization with Recombinant Replication-Defective SHtB5Ag/gJHE and SHtB5Ag/gJtat

Six-week-old Balb/C female mice were immunized intradermally with 1×10⁶ pfu of the recombinant, replication-defective SHtB5Ag/gJHE and SHtB5Ag/gJtat, or with the control virus S0Z. The inoculum was given in a volume of 100 μl. Ten days post-infection a first group of mice (n=9 mice/experimental group) were sacrificed to evaluate after priming the presence of MT and HSV1-specific T cell responses by means of IFN-γ and IL-4 Elispot assays on fresh splenocyte cultures (individual mice). The presence of TB and HSV1-specific IgM, IgG in sera was evaluated by enzyme-linked immunoassays (ELISA). At 20 or 30 days post-immunization the mice were boosted and sacrificed 6 days after the injection to evaluate after boosting the MT and HSV1-specific T cell responses by means of IFN-γ and IL-4 Elispot assays on fresh splenocyte cultures (individual mice).

### Elispot Assays

IFN-γ or IL-4 Elispot assays were carried out using the murine kits provided by Becton Dickinson, according to the manufacturer's instructions. Briefly, nitrocellulose plates were coated with 5 μg/ml of anti-IFN-γ or anti-IL-4 mAb 16 hours at 4° C. Plates were then washed with PBS and blocked with RPMI 1640 supplemented with 10% FBS for 2 hours at 37° C. Total splenocytes from individual mice (5×10⁵ cells) were added to the wells in duplicate and incubated with MT and HSV-1 derived peptides (10⁻⁶ M) for 24 hours at 37° C. Controls were represented by cells incubated with 5 μg/ml of Concanavaline A (positive control) or with medium alone (negative control). Spots were quantified using an AELVIS 4-Plate Elispot Reader (TEMA ricerca s.r.l. Bologna, Italy). The number of spots counted in the peptide-treated cultures minus the number of spots counted in the untreated cultures were the specific responses. Results are expressed as number of spot forming units (SFU)/10⁶ cells. Values at least 2-fold higher than the mean number of spots in the control wells (untreated cells) and ≧50 SFU/10⁶ cells were considered positive.

### Analysis of Antibody Responses

Anti-HSV1 or anti-TB specific antibodies in sera (IgM, IgG, IgG1 and IgG2a titers) were measured on samples collected from individual mice by enzyme-linked immunosorbent assay (ELISA) using 96-well immunoplates (Nunc Max Sorp) previously coated with TB recombinant protein (GST-TB) made in our laboratory and with100 ng/well of HSV1 viral lysate (Herpes Simplex Type 1 Purified Viral Lysate, Tebu-bio) resuspended in PBS containing 0.05% NaN₃, for 16 hours at 4° C. Plates were washed five times with PBS (pH 7.0) containing 0.05% Tween 20 (Sigma) using an automatic washer (BioRad Model 1575 ImmunoWash) and then blocked for 90 minutes at 37° C. by the addition of 200 μl/well of PBS containing 0.5% milk and 0.05% NaN₃. After extensive washes, 100 μl/well of appropriate dilutions of each serum were dispensed in duplicate wells and then incubated for 90 minutes at 37° C. Plates were washed again before the addition of 100 μl/well of horse radish peroxidase-(HRP)-conjugated goat anti-mouse IgG (Sigma), diluted 1:1000. or HRP-conjugated goat anti-mouse IgM (Sigma), diluted 1:7500. in PBS containing 0.05% Tween 20 and 1% BSA, and incubated at 37° C. for 90 minutes. In each plate, two wells were incubated with PBS containing 0.5% milk and 0.05% NaN₃ and the secondary antibodies (blank). Analysis of anti-HSV1 IgG isotype was determined using a goat anti-mouse Abs directed against IgG1 or IgG2a (Sigma), diluted 1:30000 in PBS containing 0.05% Tween 20 and 1% BSA. After incubation, plates were washed five times and subsequently a solution of 2,2′-Azinobis[3-ethylbenzothiazoline-6-sulfonic acid]-diammonium salt (ABTS) substrate (Roche Diagnostics, Mannheim-Germany) was added. The reaction was stopped with 0.1M citric acid after 50 minutes. The absorbance values were measured at 405 nm with an automatic plate reader (SUNRISE TECAN Salzburg-Austria). The cut-off value was estimated as the mean OD of 3 negative control sera plus 0.05. Each OD value was subtracted of the blank and cut-off values to obtain a net OD value. IgG titres were calculated by intercept function using the Excel program.

### Results

### Vector Construction

The cassettes containing the sequence for HIV tat protein or the sequence for tB5Ag fusion protein (Delogu, G. et al. 2002 Infection and immunity) have been introduced in plasmids containing already the HSV-1 sequences (UL41 or gJ HSV sequence) with the classical cloning procedures. More precisely, tB5Ag gene expresses a fusion protein that include several mycobacterium (Mt) antigens (TB5Ag: Ag85B, ESAT-6, Mpt 64/63/83), a TPA (tissue plasminogen activator signal sequence), which ensure the release of the fusion protein and a sequence codifying for HA epitope (haemagglutinin) that allows the identification of the protein with the specific antibody (FIG. 11). FIG. 11 shows the schematic representation of the fusion protein pTB5Ag (Delogu, G. et al. 2002 Infection and Immunity)

Plasmid pB41tB5Ag (FIG. 10) and pgJ-tat were constructed to genetically recombine with the genome of S0ZgJGFP, which is a replication-defective HSV-1 viral vector characterized by low cytotoxicity due to the deletion of both copies of one out of five immediate early (IE) ICP4 which is essential for viral replication, in the glycoprotein gJ (Us5) containing GFP and in the sequence of UL41 locus containing the LacZ gene which are not essential for viral replication and infection (FIG. 12). FIG. 12 shows a schematic representation of the SHtB5Ag/gJHE vector construction through recombination of pB41tB5Ag into UL41 locus containing the LacZ gene of S0ZgJGFP viral DNA. The recombinant viruses SHtB5Ag/gJHE and SHtB5Ag/gJTat (FIG. 13) containing the tB5Ag or tat cDNA were first identified by isolation of a clear plaque phenotype after X-gal staining or GFP screening. The viruses were purified by three rounds of limiting dilution technique and the presence of the transgenes was confirmed by Southern blot analysis (data not shown). Viral stocks of SHtB5Ag/gJHE, S0Z/gJHTat and SHtB5Ag/gJtat and of the control vector S0ZgJHE were prepared and titrated using E5 cells.

FIG. 13 shows the schematic representation of SHtB5Ag/gJHE, S0Z/gJHTat and SHtB5Ag/gJtat.

The expression of TB5Ag was controlled by Western blot analysis (data not shown). To this purpose, CB1 and Vero cells were infected with the recombinant expressing TB5Ag and expression was analysed after 12-24 hours post-infection. Uninfected and cells infected with the control vectors represented the negative controls. TB5Ag, as shown in FIG. 11, as evident from cell lysates, was expressed as the entire fusion protein (100 kDa).

Next, to determine whether Tat produced by the SHtB5Ag/gJTat vector was biologically active, HeLa3T1 cells, containing an integrated copy of the CAT reporter gene under the transcriptional control of the HIV-LTR promoter, and in which CAT expression occurs only in the presence of bioactive Tat, were infected at two different m.o.i. (0.1 and 1) with non-replicating SHtB5Ag/gJTat or SHtB5Ag/gJHE. CAT expression was measured 6, 12 and 24 hours post-infection. CAT expression was readily detected by ELISA test at 12 hours after infection, even at the lowest m.o.i. of 0.1. (FIG. 14).

FIG. 14 shows the biological activity of Tat protein.

### In Vivo Experiments

### Protocols of Immunization

### Tat-Mediated Modulation of Epitope-Specific T Cell Responses Against TB in Mice

To determine the effect of Tat on the induction of TB-specific T cell mediated responses, BALB/c female mice were immunized by intradermal injection with 2.5×10⁶ PFU of the SHtB5Ag/gJHE, SHtB5Ag/gJTat recombinant viruses or S0ZgJHE control vector. After 10 days post-infection, the presence of TB-specific T cell responses were evaluated by IFN-γ and IL4 Elispot on fresh splenocytes. To this end, 8 TB peptides were used to evaluate anti-TB T cell responses in BALB/c mice: VNYQNFAVT (SEQ ID NO:8) derived from MPT 64; FAPTNAAFD (SEQ ID NO:9) derived from MPT83; AYPITGKLG (SEQ ID NO:10) derived from MPT63; SLTKLAAAW (SEQ ID NO:11) and AYQGVQQKW (SEQ ID NO:12) derived from ESAT6; YAGSLSALL (SEQ ID NO:13), FSRPGLPVE (SEQ ID NO:14) and FQDAYNAAG (SEQ ID NO:15) derived from FBPB (Ag85B) (FIG. 15).

FIG. 15 shows the 8 TB peptides and 2 HSV were used to evaluate anti-TB and anti-HSV T cell responses respectively in BALB/c mice.

The data obtained in BALB/c mice at 10 days after infection demonstrate that the presence of Tat increases and broadens only Th1-type immune responses in comparison to the mice immunized with virus expressing TB antigens without Tat and with the S0Z virus control or PBS.

Therefore, based on these results obtained at 10 days post-infection, we made different protocols of immunization and boost using the intradermal route. 20 or 28 days after the first injection we boosted BALB/c mice with the same dose of virus used for the immunization (2.5×10⁶ PFU) and other set of groups that have received the boost at 20 days will received a third boost after 12 weeks after the first injection in order to verify the appropriate time and number of boosts and to check if the time can influence the T response against TB and HSV antigens and if there are difference in cellular response against TB and HSV, between the vectors that are expressing or not Tat. 9 mice per group are sacrificed six days after each boost.

### FIGURE LEGENDS

FIG. 1. Schematic representation of pBlueScript plasmids containing pr-lacZ or pr-tat cassettes, flanked by HSV UL41 flank sequences (A). The homologous recombination event between viral DNA of HSV1 and pB41-lacZ or pB41-tat plasmids resulted in the generation of the HSV1-LacZ or HSV1-Tat recombinant viruses (B). The black square symbolises the UL41 locus (vhs gene) that has been deleted in the HSV backbone. The white squares symbolise the terminal and internal repeats of the HSV genome delimiting the unique regions (U_{L}: unique long; U_{S}: unique short). Analysis of Tat expression by HSV-Tat recombinant vector (C). Western blot analysis of BALB/c cells infected with HSV1-Tat (1 m.o.i) after 12 and 24 hours post-infection. Controls are represented by uninfected cells and by cells infected with wild type HSV1 or HSV1-LacZ. Recombinant Tat protein (Tat) has been used as positive control.

FIG. 2. Analysis of HSV1-specific T cell responses in C57BL/6 mice. Mice (n=15) were immunised intravaginally with 10³ pfu of live attenuated HSV1-Tat or HSV-LacZ. Seven days after infection, splenocytes were isolated from 5 mice/group and tested by IFN-γ (A) and IL4 (B) ELISPOT assays. Results are expressed as number of spot forming units (SFU)/million cells. Values at least 2-fold higher than the mean number of spots in the control wells (untreated cells) and ≧50 SFU/million cells were considered positive. The mean values (+SD) of five mice are reported. The results of one representative experiment (out of three) are shown.

FIG. 3. Analysis of HSV1-specific T cell responses in BALB/c mice. Mice (n=15) were immunised intravaginally with 10³ pfu of live attenuated HSV1-Tat or HSV-LacZ. Seven days after infection, splenocytes were isolated from 5 mice/group and tested by IFN-γ (A) and IL4 (B) ELISPOT assays after ex vivo re-stimulation for 5 days with HSV1 peptides. Results are expressed as number of spot forming units (SFU)/million cells. Values at least 2-fold higher than the mean number of spots in the control wells (untreated cells) and ≧50 SFU/10⁶ cells were considered positive. The mean values (+SD) of five mice are reported. The results of one representative experiment (out of three) are shown.

FIG. 4. Analysis of HSV1-specific T cell responses in C57BL/6 mice. Mice (n=10) were immunised intradermally with 10³ pfu of live attenuated HSV1-Tat or HSV-LacZ. Seven days after infection, fresh splenocytes from 5 mice/group were tested by IFN-γ (A) and IL4 (B) ELISPOT against HSV1 epitopes. Results are expressed as number of spot forming units (SFU)/million cells. Values at least 2-fold higher than the mean number of spots in the control wells (untreated cells) and ≧50 SFU/10⁶ cells were considered positive. The mean values (+SD) of five mice are reported. The results of one representative experiment (out of three) are shown.

FIG. 5. Evaluation of anti-HSV1 specific antibody titres (IgG, IgG1, IgG2a) in sera of individual mice treated with HSV1-Tat or HSV1-LacZ. Mice (n=15) were immunised intravaginally with 10³ pfu of live attenuated HSV1-Tat or HSV-LacZ. Twenty days after infection, anti-HSV1 antibody responses were measured by ELISA in sera collected by eye-bleeding from 5 mice/group. The results of one representative experiment (out of three) are shown. C57BL/6 (A) and BALB/c (B) mice.

FIG. 6. Survival of BALB/c and C57BL/6 mice treated with HSV1-LacZ or HSV1-Tat following challenge with a lethal dose of HSV1. Mice (n=15) were immunised intravaginally with 10³ pfu of HSV1-LacZ or HSV1-Tat, as described in FIGS. 3 and 4, and 28 days later challenged (n=10) intravaginally with a lethal dose of wild-type HSV1 (1.5×10⁶ pfu/mouse for BALB/c mice and 1.5×10⁸ pfu/mouse for C57BL/6 mice). Mice were observed daily for appearance of HSV1 signs of disease an death. The results of one representative experiment (out of three) are shown. (A) BALB/c mice, (B) C57BL/6 mice.

FIG. 7. Survival of C57BL/6 mice treated with HSV1-LacZ or HSV1-Tat following challenge with a lethal dose of HSV1. Mice (n=10) were immunised intradermally with 10³ pfu of HSV1-LacZ or HSV1-Tat, as described in FIG. 5, and 28 days later challenged (n=5) intravaginally with a lethal dose of wild-type HSV1 (1.5×10⁸ pfu/mouse). Mice were observed daily for appearance of HSV1 signs of disease and death.

FIG. 8. Schematic representation of BAC-HSVLuc genome (A). Schematic representation of HSVLucΔ27 vector (B). Schematic representation of HSVLucΔ27gJHE vector (C). Schematic representation of HSVLucΔ27gJTat vector (D).

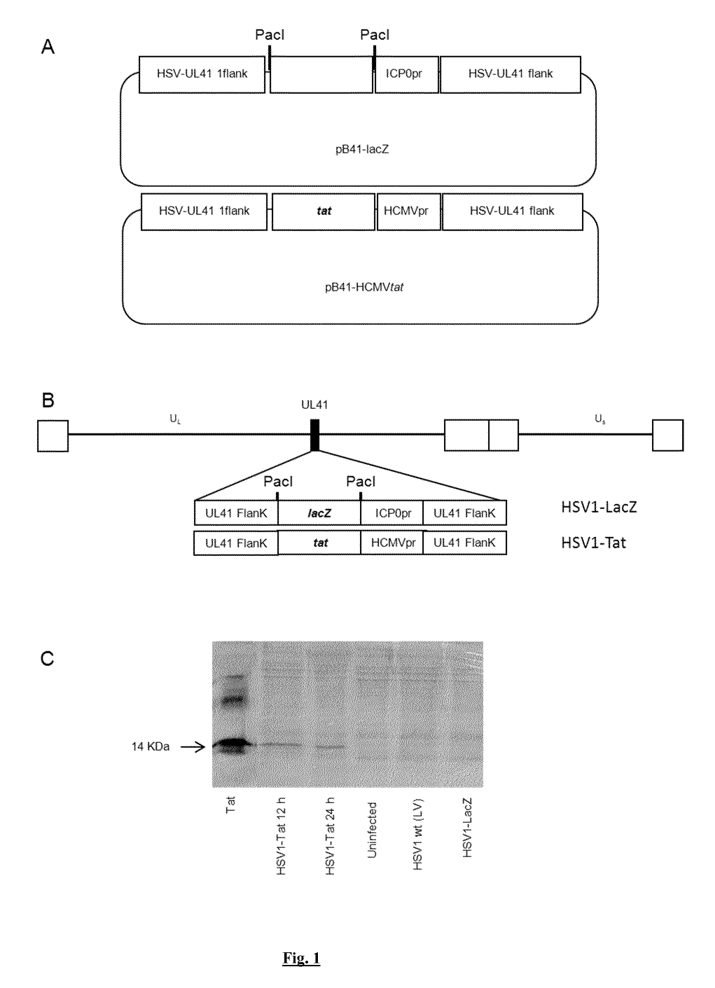

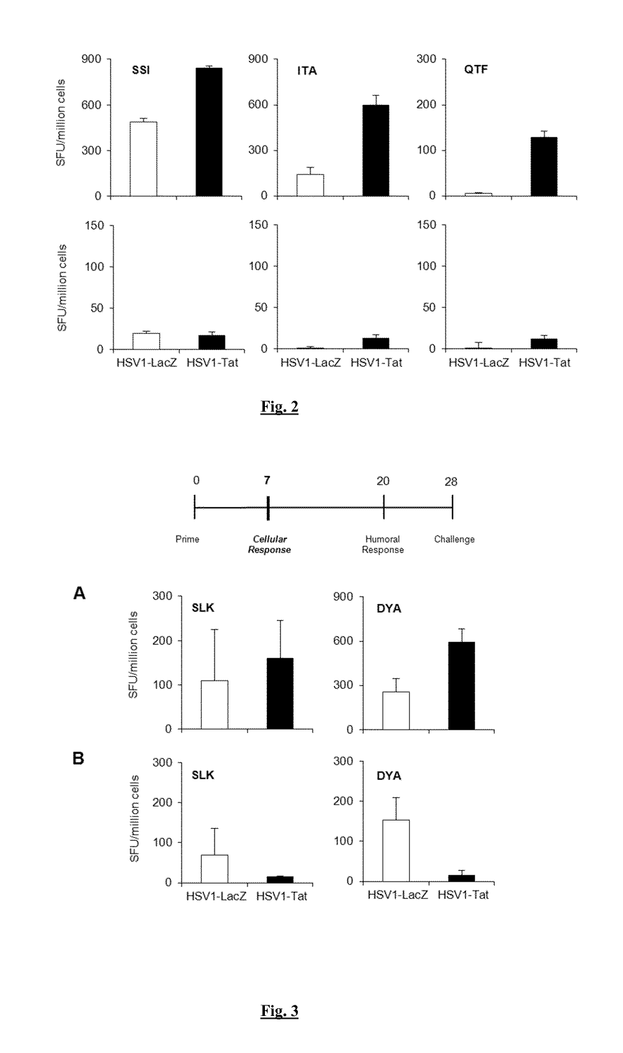

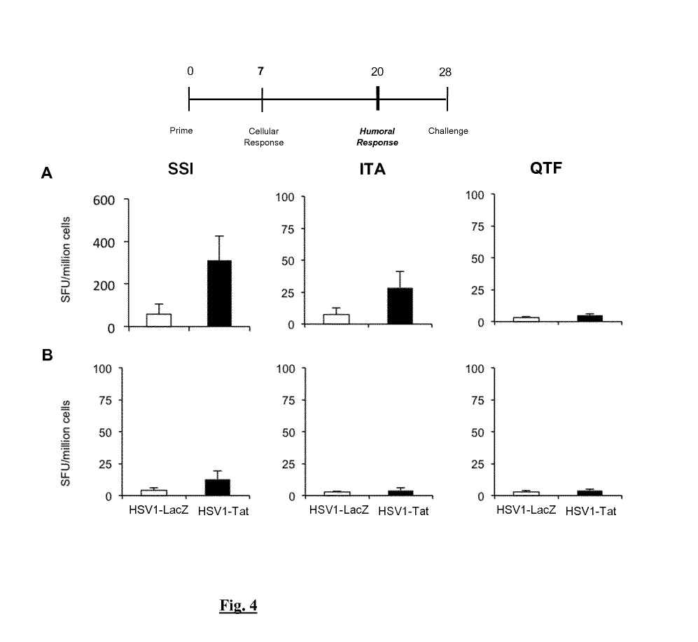

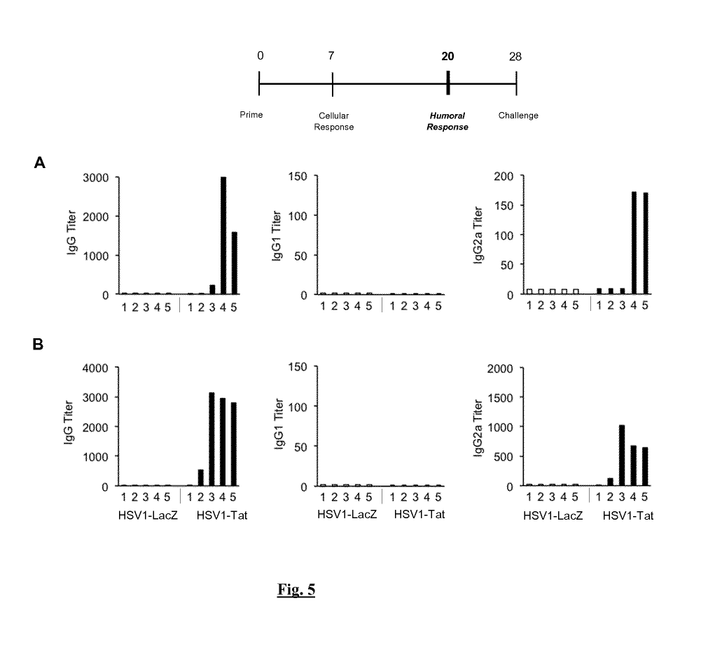

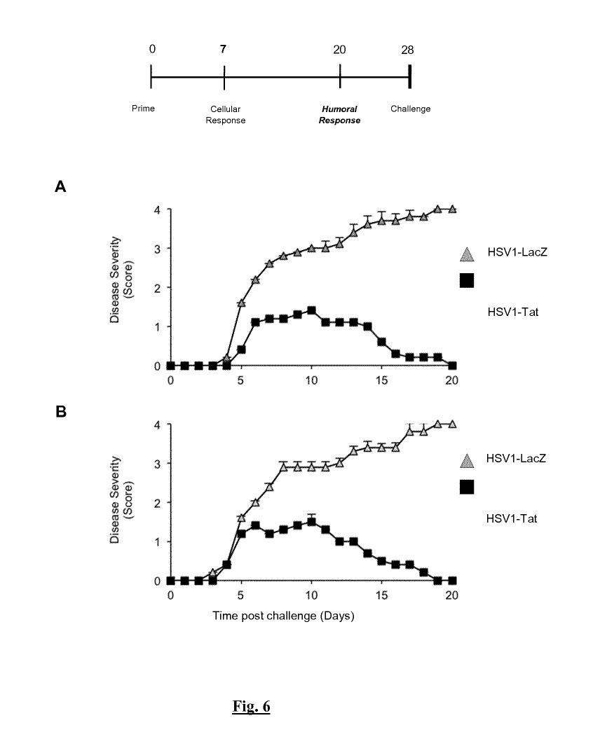

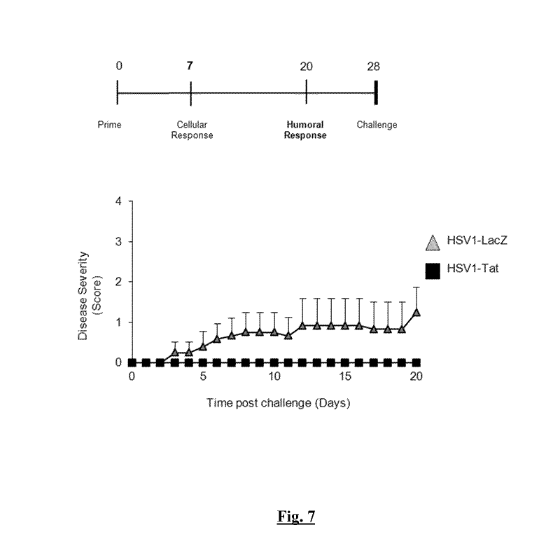

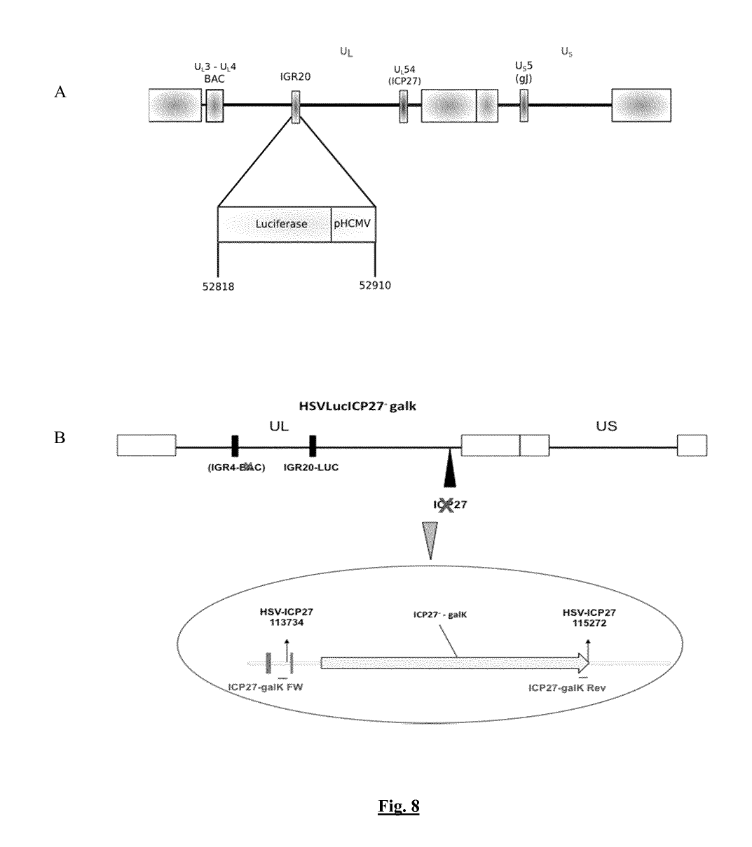

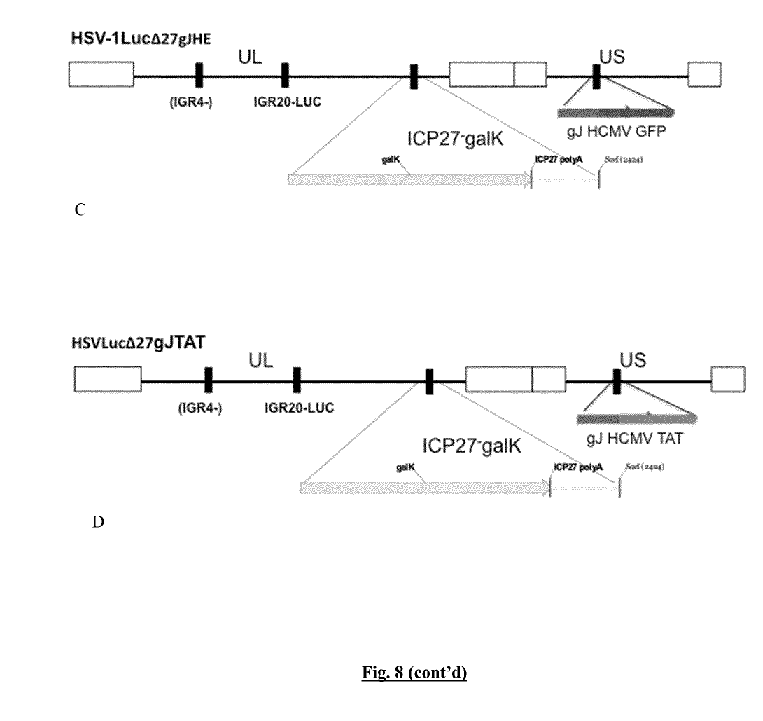

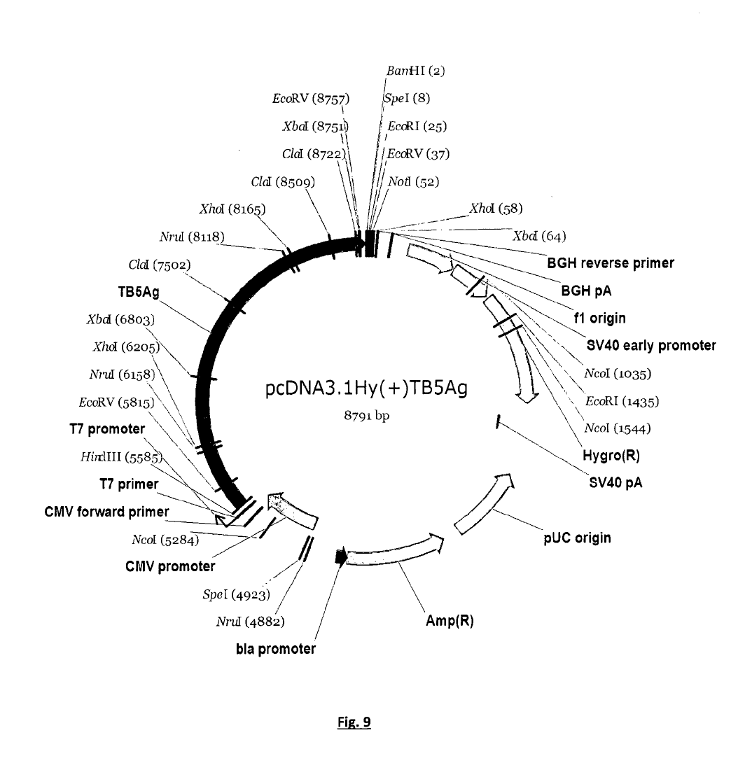

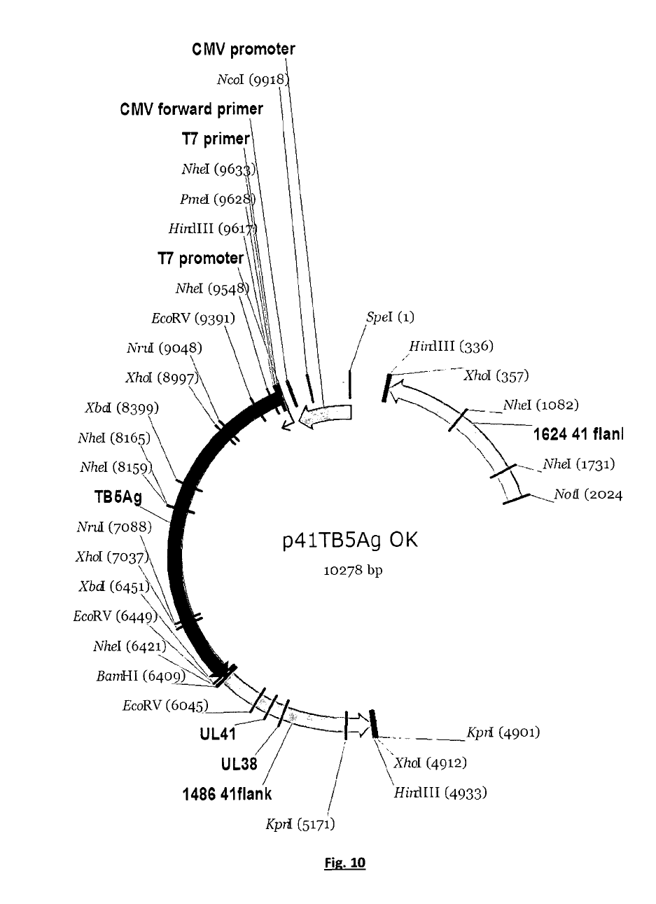

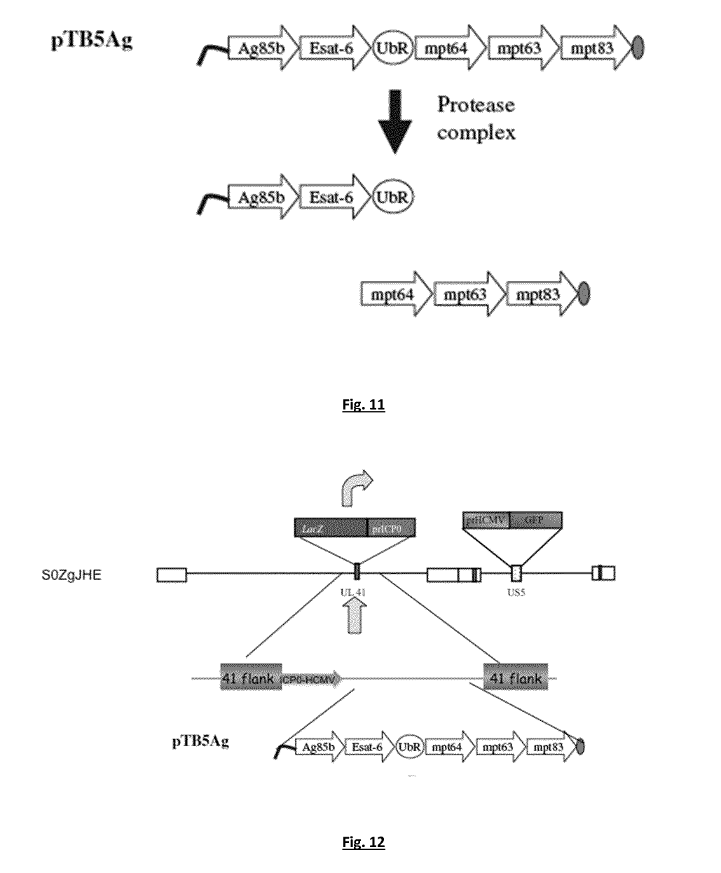

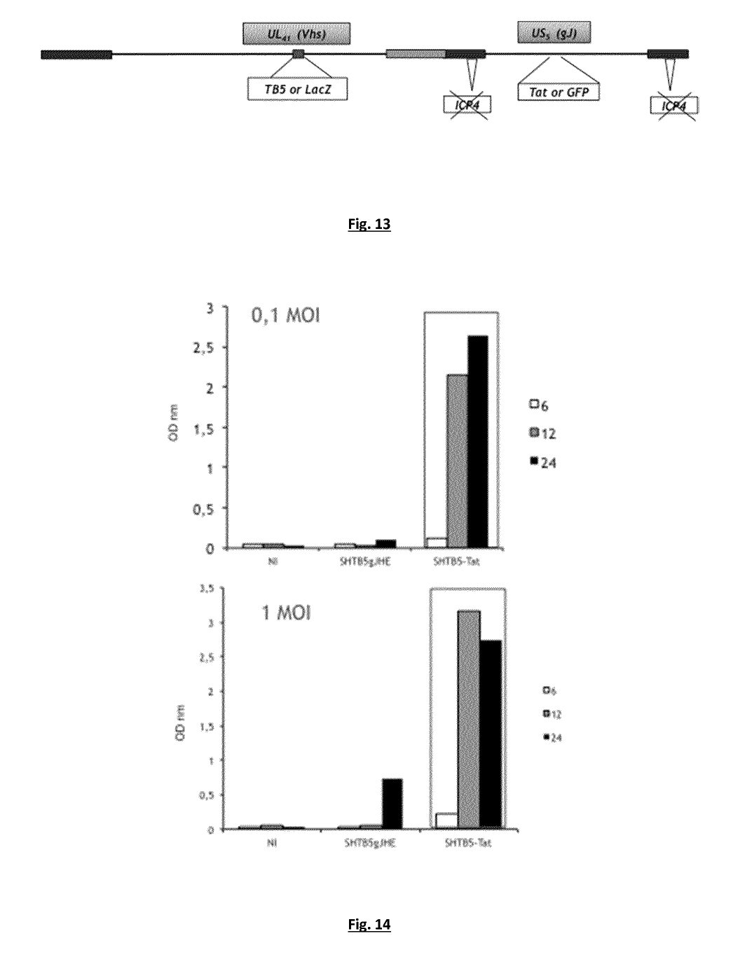

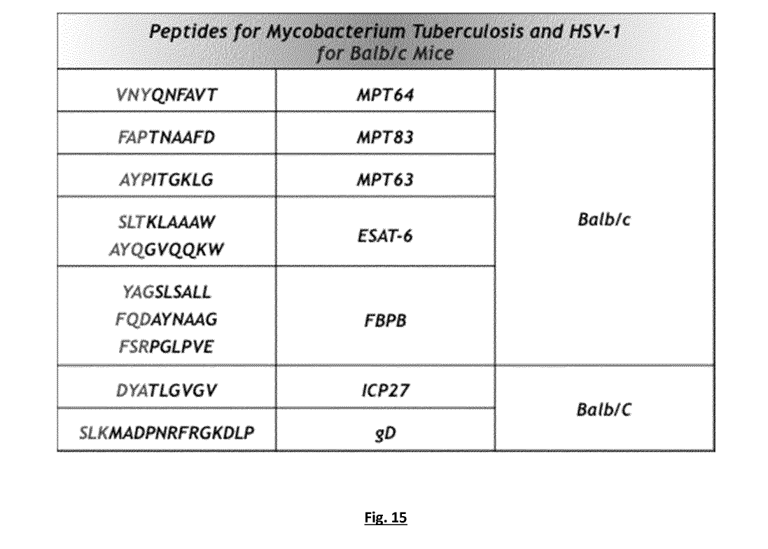

### REFERENCES

1. Chentoufi, A. A., N. R. Binder, N. Berka, G. Durand, A. Nguyen, I. Bettahi, B. Maillere, and L. BenMohamed. 2008. Asymptomatic human CD4+ cytotoxic T-cell epitopes identified from herpes simplex virus glycoprotein B. Journal of virology 82:11792-11802.
2. Dasgupta, G., A. B. Nesburn, S. L. Wechsler, and L. BenMohamed. 2010. Developing an asymptomatic mucosal herpes vaccine: the present and the future. Future Microbiol 5:1-4.
3. Huilan, Y., Z. Cui, F. Jianyong, G. Lei, and Q. Wei. 2010. Construction of, and T-helper (Th)1/Th2 immune responses to, a herpes simplex virus type 2 glycoprotein D-cytotoxic T-lymphocyte epitope DNA vaccine. Clin Exp Dermatol 35:537-542.
4. Akhrameyeva, N. V., P. Zhang, N. Sugiyama, S. M. Behar, and F. Yao. 2011. Development of a glycoprotein D-expressing dominant-negative and replication-defective herpes simplex virus 2 (HSV-2) recombinant viral vaccine against HSV-2 infection in mice. Journal of virology 85:5036-5047.
5. Dupuis, M., K. Denis-Mize, A. LaBarbara, W. Peters, I. F. Charo, D. M. McDonald, and G. Ott.

2001. Immunization with the adjuvant M F59 induces macrophage trafficking and apoptosis. Eur J Immunol31:2910-2918.
6. Ugozzoli, M., D. T. O'Hagan, and G. S. Ott. 1998. Intranasal immunization of mice with herpes simplex virus type 2 recombinant gD2: the effect of adjuvants on mucosal and serum antibody responses. Immunology 93:563-571.
7. Duerst, R. J., and L. A. Morrison. 2003. Innate immunity to herpes simplex virus type 2. Viral Immunol 16:475-490.
8. Rouse, B. T., and M. Gierynska. 2001. Immunity to herpes simplex virus: a hypothesis. Herpes 8 Suppl 1:2A-5A.
9. Khanna, K. M., A. J. Lepisto, and R. L. Hendricks. 2004. Immunity to latent viral infection: many skirmishes but few fatalities. Trends Immunol 25:230-234.
10. Khanna, K. M., A. J. Lepisto, V. Decman, and R. L. Hendricks. 2004. Immune control of herpes simplex virus during latency. Curr Opin Immunol 16:463-469.
11. Perng, G. C., and C. Jones. 2010. Towards an understanding of the herpes simplex virus type 1 latency-reactivation cycle. Interdiscip Perspect Infect Dis 2010:262415.
12. Day, C. L., D. E. Kaufmann, P. Kiepiela, J. A. Brown, E. S. Moodley, S. Reddy, E. W. Mackey, J. D. Miller, A. J. Leslie, C. DePierres, Z. Mncube, J. Duraiswamy, B. Zhu, Q. Eichbaum, M. Altfeld, E. J. Wherry, H. M. Coovadia, P. J. Goulder, P. Klenerman, R. Ahmed, G. J. Freeman, and B. D. Walker. 2006. PD-1 expression on HIV-specific T cells is associated with T-cell exhaustion and disease progression. Nature 443:350-354.
13. Giorgi, J. V., L. E. Hultin, J. A. McKeating, T. D. Johnson, B. Owens, L. P. Jacobson, R. Shih, J. Lewis, D. J. Wiley, J. P. Phair, S. M. Wolinsky, and R. Detels. 1999. Shorter survival in advanced human immunodeficiency virus type 1 infection is more closely associated with T lymphocyte activation than with plasma virus burden or virus chemokine coreceptor usage. The Journal of infectious diseases 179:859-870.
14. Sodora, D. L., and G. Silvestri. 2008. Immune activation and AIDS pathogenesis. AIDS (London, England) 22:439-446.
15. Haas, A., K. Zimmermann, and A. Oxenius. 2011. Antigen-dependent and -independent mechanisms of T and B cell hyperactivation during chronic HIV-1 infection. Journal of virology 85:12102-12113.
16. Faller, E. M., S. M. Sugden, M. J. McVey, J. A. Kakal, and P. A. MacPherson. 2010. Soluble HIV Tat protein removes the IL-7 receptor alpha-chain from the surface of resting CD8 T cells and targets it for degradation. J Immunol 185:2854-2866.
17. Fanales-Belasio, E., S. Moretti, V. Fiorelli, A. Tripiciano, M. R. Pavone Cossut, A. Scoglio, B. Collacchi, F. Nappi, I. Macchia, S. Bellino, V. Francavilla, A. Caputo, G. Barillari, M. Magnani, M. E. Laguardia, A. Cafaro, F. Titti, P. Monini, F. Ensoli, and B. Ensoli. 2009. HIV-1 Tat addresses dendritic cells to induce a predominant Th1-type adaptive immune response that appears prevalent in the asymptomatic stage of infection. J Immunol 182:2888-2897.
18. Caputo, A., R. Gavioli, S. Bellino, 0. Longo, A. Tripiciano, V. Francavilla, C. Sgadari, G. Paniccia, F. Titti, A. Cafaro, F. Ferrantelli, P. Monini, F. Ensoli, and B. Ensoli. 2009. HIV-1 Tat-based vaccines: an overview and perspectives in the field of HIV/AIDS vaccine development. Int Rev Immunol 28:285-334.
19. Gavioli, R., S. Cellini, A. Castaldello, R. Voltan, E. Gallerani, F. Gagliardoni, C. Fortini, E. B. Cofano, C. Triulzi, A. Cafaro, I. Srivastava, S. Barnett, A. Caputo, and B. Ensoli. 2008. The Tat protein broadens T cell responses directed to the HIV-1 antigens Gag and Env: implications for the design of new vaccination strategies against AIDS. Vaccine 26:727-737.
20. Sui, Z., L. F. Sniderhan, G. Schifitto, R. P. Phipps, H. A. Gelbard, S. Dewhurst, and S. B. Maggirwar. 2007. Functional synergy between CD40 ligand and HIV-1 Tat contributes to inflammation: implications in HIV type 1 dementia. J Immunol 178:3226-3236.
21. Gavioli, R., E. Gallerani, C. Fortini, M. Fabris, A. Bottoni, A. Canella, A. Bonaccorsi, M. Marastoni, F. Micheletti, A. Cafaro, P. Rimessi, A. Caputo, and B. Ensoli. 2004. HIV-1 tat protein modulates the generation of cytotoxic T cell epitopes by modifying proteasome composition and enzymatic activity. J Immunol 173:3838-3843.
22. Fanales-Belasio, E., S. Moretti, F. Nappi, G. Barillari, F. Micheletti, A. Cafaro, and B. Ensoli. 2002. Native HIV-1 Tat protein targets monocyte-derived dendritic cells and enhances their maturation, function, and antigen-specific T cell responses. J Immunol 168:197-206.
23. Zagury, D., A. Lachgar, V. Chams, L. S. Fall, J. Bernard, J. F. Zagury, B. Bizzini, A. Gringeri, E. Santagostino, J. Rappaport, M. Feldman, A. Burny, and R. C. Gallo. 1998. Interferon alpha and Tat involvement in the immunosuppression of uninfected T cells and C—C chemokine decline in AIDS. Proceedings of the National Academy of Sciences of the United States of America 95:3851-3856.
24. Ott, M., S. Emiliani, C. Van Lint, G. Herbein, J. Lovett, N. Chirmule, T. McCloskey, S. Pahwa, and E. Verdin. 1997 Immune hyperactivation of HIV-1-infected T cells mediated by Tat and the CD28 pathway. Science (New York, N.Y. 275:1481-1485.
25. Zauli, G., D. Gibellini, C. Celeghini, C. Mischiati, A. Bassini, M. La Placa, and S. Capitani. 1996. Pleiotropic effects of immobilized versus soluble recombinant HIV-1 Tat protein on CD3-mediated activation, induction of apoptosis, and HIV-1 long terminal repeat transactivation in purified CD4+ T lymphocytes. J Immunol 157:2216-2224.
26. Westendorp, M. O., M. Li-Weber, R. W. Frank, and P. H. Krammer 1994. Human immunodeficiency virus type 1 Tat upregulates interleukin-2 secretion in activated T cells. Journal of virology 68:4177-4185.
27. Viscidi, R. P., K. Mayur, H. M. Lederman, and A. D. Frankel. 1989. Inhibition of antigen-induced lymphocyte proliferation by Tat protein from HIV-1. Science (New York, N. Y 246:1606-1608.
28. Ensoli, B., S. Bellino, A. Tripiciano, O. Longo, V. Francavilla, S. Marcotullio, A. Cafaro, O. Picconi, G. Paniccia, A. Scoglio, A. Arancio, C. Ariola, M. J. Ruiz Alvarez, M. Campagna, D. Scaramuzzi, C. Iori, R. Esposito, C. Mussini, F. Ghinelli, L. Sighinolfi, G. Palamara, A. Latini, G. Angarano, N. Ladisa, F. Soscia, V. S. Mercurio, A. Lazzarin, G. Tambussi, R. Visintini, F. Mazzotta, M. Di Pietro, M. Galli, S. Rusconi, G. Carosi, C. Torti, G. Di Perri, S. Bonora, F. Ensoli, and E. Garaci. 2010. Therapeutic immunization with HIV-1 Tat reduces immune activation and loss of regulatory T-cells and improves immune function in subjects on HAART. PLoS One 5:e13540.
29. Li, J. C., H. C. Yim, and A. S. Lau. 2010. Role of HIV-1 Tat in AIDS pathogenesis: its effects on cytokine dysregulation and contributions to the pathogenesis of opportunistic infection. AIDS (London, England) 24:1609-1623.
30. Rezza, G., V. Fiorelli, M. Dorrucci, M. Ciccozzi, A. Tripiciano, A. Scoglio, B. Collacchi, M.

Ruiz-Alvarez, C. Giannetto, A. Caputo, L. Tomasoni, F. Castelli, M. Sciandra, A. Sinicco, F. Ensoli, S. Butto, and B. Ensoli. 2005. The presence of anti-Tat antibodies is predictive of long-term nonprogression to AIDS or severe immunodeficiency: findings in a cohort of HIV-1 seroconverters. The Journal of infectious diseases 191:1321-1324.
31. Re, M. C., D. Gibellini, F. Vitone, and M. La Placa. 2001. Antibody to HIV-1 Tat protein, a key molecule in HIV-1 pathogenesis. A brief review. The new microbiologica 24:197-205.
32. Herbeuval, J. P., J. Nilsson, A. Boasso, A. W. Hardy, M. J. Kruhlak, S. A. Anderson, M. J. Dolan, M. Dy, J. Andersson, and G. M. Shearer. 2006. Differential expression of IFN-alpha and TRAIL/D R5 in lymphoid tissue of progressor versus nonprogressor HIV-1-infected patients. Proceedings of the National Academy of Sciences of the United States of America 103:7000-7005.
33. Brenchley, J. M., D. A. Price, T. W. Schacker, T. E. Asher, G. Silvestri, S. Rao, Z. Kazzaz, E. Bornstein, O. Lambotte, D. Altmann, B. R. Blazar, B. Rodriguez, L. Teixeira-Johnson, A. Landay, J. N. Martin, F. M. Hecht, L. J. Picker, M. M. Lederman, S. G. Deeks, and D. C. Douek. 2006. Microbial translocation is a cause of systemic immune activation in chronic HIV infection. Nature medicine 12:1365-1371.
34. Chang, H. C., F. Samaniego, B. C. Nair, L. Buonaguro, and B. Ensoli. 1997. HIV-1 Tat protein exits from cells via a leaderless secretory pathway and binds to extracellular matrix-associated heparan sulfate proteoglycans through its basic region. AIDS (London, England) 11:1421-1431.
35. Krisky, D. M., P. C. Marconi, T. J. Oligino, R. J. Rouse, D. J. Fink, J. B. Cohen, S. C. Watkins, and J. C. Glorioso. 1998. Development of herpes simplex virus replication-defective multigene vectors for combination gene therapy applications. Gene Ther 5:1517-1530.
36. Marconi, P., D. Krisky, T. Oligino, P. L. Poliani, R. Ramakrishnan, W. F. Goins, D. J. Fink, and J. C. Glorioso. 1996. Replication-defective herpes simplex virus vectors for gene transfer in vivo. Proceedings of the National Academy of Sciences of the United States of America 93:11319-11320.
37. Gadzinski, A., D. Matz, E. Favre, and M. Leonetti. Transfer of the ability of HIV-1 Tat to raise an adjuvant-free humoral immune response to unrelated antigens. Vaccine 30:2859-2868.
38. Kittiworakarn, J., A. Lecoq, G. Moine, R. Thai, E. Lajeunesse, P. Drevet, C. Vidaud, A. Menez, and M. Leonetti. 2006. HIV-1 Tat raises an adjuvant-free humoral immune response controlled by its core region and its ability to form cysteine-mediated oligomers. J Biol Chem 281:3105-3115.
39. Kronenberg, K., S. Brosch, F. Butsch, Y. Tada, N. Shibagaki, M. C. Udey, and E. von Stebut. 2010. Vaccination with TAT-antigen fusion protein induces protective, CD8(+) T cell-mediated immunity against Leishmania major. The Journal of investigative dermatology 130:2602-2610.
40. Kulkarni, A., D. S. Ravi, K. Singh, S. Rampalli, V. Parekh, D. Mitra, and S. Chattopadhyay. 2005. HIV-1 Tat modulates T-bet expression and induces Th1 type of immune response. Biochemical and biophysical research communications 329:706-712.
41. Nguyen, H. V., E. Mouly, K. Chemin, R. Luinaud, R. Despres, J. P. Fermand, B. Amulf, and J. C. Bories. 2012. The Ets-1 transcription factor is required for Stat1-mediated T-bet expression and IgG2a class switching in mouse B cells. Blood 119:4174-4181.
42. Peng, S. L., S. J. Szabo, and L. H. Glimcher. 2002. T-bet regulates IgG class switching and pathogenic autoantibody production. Proceedings of the National Academy of Sciences of the United States of America 99:5545-5550.
43. Dasgupta, G., A. A. Chentoufi, A. B. Nesburn, S. L. Wechsler, and L. BenMohamed. 2009. New concepts in herpes simplex virus vaccine development: notes from the battlefield. Expert Rev Vaccines 8:1023-1035.
44. Roizman, B. 1996. The function of herpes simplex virus genes: a primer for genetic engineering of novel vectors. Proceedings of the National Academy of Sciences of the United States of America 93:11307-11312.
45. Manservigi, R., R. Argnani, and P. Marconi. 2010. HSV Recombinant Vectors for Gene Therapy. Open Virol J4:123-156.
46. Watanabe, D. 2010. Medical application of herpes simplex virus. J Dermatol Sci 57:75-82.
47. Whitley, R. J., and B. Roizman. 2002. Herpes simplex viruses: is a vaccine tenable? J Clin Invest 110:145-151.
48. Cotter, C. R., M. L. Nguyen, J. S. Yount, C. B. Lopez, J. A. Blaho, and T. M. Moran. 2010. The virion host shut-off (vhs) protein blocks a TLR-independent pathway of herpes simplex virus type 1 recognition in human and mouse dendritic cells. PLoS One 5:e8684.
49. Smiley, J. R. 2004. Herpes simplex virus virion host shutoff protein: immune evasion mediated by a viral RNase? Journal of virology 78:1063-1068.
50. Samady, L., E. Costigliola, L. MacCormac, Y. McGrath, S. Cleverley, C. E. Lilley, J. Smith, D. S. Latchman, B. Chain, and R. S. Coffin. 2003. Deletion of the virion host shutoff protein (vhs) from herpes simplex virus (HSV) relieves the viral block to dendritic cell activation: potential of vhs-HSV vectors for dendritic cell-mediated immunotherapy. Journal of virology 77:3768-3776.
51. Hill, A., P. Jugovic, I. York, G. Russ, J. Bennink, J. Yewdell, H. Ploegh, and D. Johnson. 1995. Herpes simplex virus turns off the TAP to evade host immunity. Nature 375:411-415.
52. Smibert, C. A., and J. R. Smiley. 1990. Differential regulation of endogenous and transduced beta-globin genes during infection of erythroid cells with a herpes simplex virus type 1 recombinant. Journal of virology 64:3882-3894.
53. Read, G. S., and N. Frenkel. 1983. Herpes simplex virus mutants defective in the virion-associated shutoff of host polypeptide synthesis and exhibiting abnormal synthesis of alpha (immediate early) viral polypeptides. Journal of virology 46:498-512.
54. Strelow, L. I., and D. A. Leib. 1995. Role of the virion host shutoff (vhs) of herpes simplex virus type 1 in latency and pathogenesis. Journal of virology 69:6779-6786.
55. Strelow, L., T. Smith, and D. Leib. 1997. The virion host shutoff function of herpes simplex virus type 1 μlays a role in corneal invasion and functions independently of the cell cycle. Virology 231:28-34.
56. Smith, T. J., L. A. Morrison, and D. A. Leib. 2002. Pathogenesis of herpes simplex virus type 2 virion host shutoff (vhs) mutants. Journal of virology 76:2054-2061.
57. Brockman, M. A., and D. M. Knipe. 2002. Herpes simplex virus vectors elicit durable immune responses in the presence of preexisting host immunity. Journal of virology 76:3678-3687.
58. Haluszczak, C., A. D. Akue, S. E. Hamilton, L. D. Johnson, L. Pujanauski, L. Teodorovic, S. C. Jameson, and R. M. Kedl. 2009. The antigen-specific CD8+ T cell repertoire in unimmunized mice includes memory phenotype cells bearing markers of homeostatic expansion. J Exp Med 206:435-448.
59. Krisky, D. M., P. C. Marconi, T. Oligino, R. J. Rouse, D. J. Fink, and J. C. Glorioso. 1997. Rapid method for construction of recombinant HSV gene transfer vectors. Gene Ther 4:1120-1125.
60. Arya, S. K., C. Guo, S. F. Josephs, and F. Wong-Staal. 1985. Trans-activator gene of human T-lymphotropic virus type III (HTLV-III). Science (New York, N.Y. 229:69-73.
61. Fraefel, C., P. Marconi, and A. L. Epstein. 2011. Herpes simplex virus type 1-derived recombinant and amplicon vectors. Methods Mol Biol 737:303-343.
62. Mineta, T., S. D. Rabkin, T. Yazaki, W. D. Hunter, and R. L. Martuza. 1995. Attenuated multi-mutated herpes simplex virus-1 for the treatment of malignant gliomas. Nature medicine 1:938-943.
63. Whitley, R. J., E. R. Kern, S. Chatterjee, J. Chou, and B. Roizman 1993. Replication, establishment of latency, and induced reactivation of herpes simplex virus gamma 1 34.5 deletion mutants in rodent models. J Clin Invest 91:2837-2843.
64. Chou, J., E. R. Kern, R. J. Whitley, and B. Roizman. 1990. Mapping of herpes simplex virus-1 neurovirulence to gamma 134.5, a gene nonessential for growth in culture. Science (New York, 1V. Y250:1262-1266.
65. Posavad C M, Remington M, Mueller D E, Zhao L, Magaret A S, et al. 2010. Detailed characterization of T cell responses to herpes simplex virus-2 in immune seronegative persons. J Immunol 184: 3250-3259.
66. Tanaka M, Kagawa H, Yamanashi Y, Sata T, Kawaguchi Y 2003. Construction of an excisable bacterial artificial chromosome containing a full-length infectious clone of herpes simplex virus type 1: viruses reconstituted from the clone exhibit wild-type properties in vitro and in vivo. J Virol 77: 1382-1391.
67. Warming S, Costantino N, Court D L, Jenkins N A, Copeland N G 2005. Simple and highly efficient BAC recombineering using galK selection. Nucleic Acids Res 33: e36.
68. Krisky D M, Marconi P C, Oligino T, Rouse R J, Fink D J, et al. 1997. Rapid method for construction of recombinant HSV gene transfer vectors. Gene Ther 4: 1120-1125.
69. Fraefel C, Marconi P, Epstein A L 2011. Herpes simplex virus type 1-derived recombinant and amplicon vectors. Methods Mol Biol 737: 303-343.

1. A vaccine for a DNA virus, comprising an expression vector for HIV1 Tat, wherein the vector is a compromised form of said DNA virus.

2. The vaccine of claim 1, wherein said compromised form is an attenuated or avirulent form.

3. The vaccine of claim 1, wherein the DNA virus is selected from the group consisting of: the order Caudovirales, including the families Myoviridae, Podoviridae, Siphoviridae; the order Herpesvirales, including the families Alloherpesvirdae, Herpersviridae, and Malacoherpesviridae; the order Ligamenvirales, including the families Lipothrixviridae, and Rudiviridae; and the families; Adenovirdae, Ampullaviridae, Ascoviridae, Asfarviridae, Baculoviridae, Bicaudaviridae, Clavaviridae, Corticoviridae, Fuselloviridae, Globuloviridae, Guttaviridae, Hytrosaviridae, Iridoviridae, Miminviridae, Nimaviridae, Papillomaviridae, Phycodnaviridae, Plasmaviridae, Polydnaviruses, Polyomaviridae, Poxviridae, and Tectiviridae; a preferred order being the Herpesvirales, a preferred family being the Herpesviridae, and a preferred virus being herpes simplex virus type 1, or HSV1.

4. The vaccine of claim 1, wherein DNA encoding HIV1 Tat is integrated into a location on the viral genome such as to disrupt a significant function, or protein of the virus, especially one that is necessary for virulence, genome integration, and/or baffling the host immune system, especially the viral host shutoff protein (vhs).

5. The vaccine of claim 1, wherein the vector is a form of the virus and is encapsulated.

6. The vaccine of claim 1, wherein said Tat is encoded by a cassette comprising the Tat-encoding sequence and a promoter therefor, said expression cassette being suitable to ensure the expression of the tat gene within the target cell to produce Tat in a biologically active form.

7. The vaccine of claim 1, wherein the virus is an avirulent form selected from replication-deficient forms of the virus, attenuated forms of the virus, and forms of the virus modified, to prevent integration of the viral genome into the host genome.

8. The vaccine of claim 1, wherein the vector encodes one or more further functions or proteins.

9. The vaccine of claim 1, wherein the vector encodes one or more further immunogens from at least one further infectious agent.

10. The vaccine of claim 9, wherein the vector encodes at least one antigen from the group consisting of: Bacillus anthracis, Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis and Enterococcus faecium, Escherichia coli (generally), Enterotoxigenic Escherichia coli (ETEC), Enteropathogenic E. coli, E. coli O157:H7, Francisella tularensis, Haemophilus influenzae, Helicobacter pylori, Legionella pneumophila, Leptospira interrogans, Listeria monocytogenes, Mycobacterium leprae, Mycobacterium tuberculosis, Mycoplasma pneumoniae, Neisseria gonorrhoeae, Neisseria meningitidis, Pseudomonas aeruginosa, Rickettsia rickettsii, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Vibrio cholerae, and Yersinia pestis.

11. The vaccine of claim 10, wherein the vector encodes one or more mycobacterial antigens selected from ESAT6, Ag85B, and Mpt64-63-83.

12. The vaccine of claim 9, wherein said DNA virus is HSV.

13. An expression vector for HIV1 Tat, wherein the vector is a compromised form of said DNA virus.

14. A method of treating or preventing a condition associated with a DNA virus comprising administering an effective amount of the vector of claim 16 to a patient in need thereof.

15. A method of treating a condition associated with a DNA virus comprising administrating an effective amount of the vaccine of claim 1 to a patient in need thereof.


SEQUENCE LISTING (15 sequences)

SEQ ID NO: 1 - DNA, 261 bp, Human immunodeficiency virus type 1
atggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagacctcctcaaggcagtcagactcatcaagtttctctatcaaagcaacccacctcccaatcccgaggggacccgacaggcccgaaggaatag

SEQ ID NO: 2 - protein, 86 aa, Human immunodeficiency virus type 1
MEPVDPRLEPWKHPGSQPKTACTNCYCKKCCFHCQVCFITKALGISYGRKKRRQRRRPPQGSQTHQVSLSKQPTSQSRGDPTGPKE

SEQ ID NO: 3 - protein, 8 aa, Herpes Simplex Virus Type 1
SSIEFARL

SEQ ID NO: 4 - protein, 8 aa, Herpes Simplex Virus Type 1
ITAYGLVL

SEQ ID NO: 5 - protein, 8 aa, Herpes Simplex Virus Type 1
QTFDFGRL

SEQ ID NO: 6 - protein, 9 aa, Herpes Simplex Virus Type 1
DYATLGVGV

SEQ ID NO: 7 - protein, 16 aa, Herpes Simplex Virus Type 1
SLKMADPNRFRGKDLP

SEQ ID NO: 8 - protein, 9 aa, Mycobacterium tuberculosis
VNYQNFAVT

SEQ ID NO: 9 - protein, 9 aa, Mycobacterium tuberculosis
FAPTNAAFD

SEQ ID NO: 10 - protein, 9 aa, Mycobacterium tuberculosis
AYPITGKLG

SEQ ID NO: 11 - protein, 9 aa, Mycobacterium tuberculosis
SLTKLAAAW

SEQ ID NO: 12 - protein, 9 aa, Mycobacterium tuberculosis
AYQGVQQKW

SEQ ID NO: 13 - protein, 9 aa, Mycobacterium tuberculosis
YAGSLSALL

SEQ ID NO: 14 - protein, 9 aa, Mycobacterium tuberculosis
FSRPGLPVE

SEQ ID NO: 15 - protein, 9 aa, Mycobacterium tuberculosis
FQDAYNAAG